(19) United States
(12) Patent Application Publication
(10) Pub. No.: US 2009/0016971 A1
(43) Pub. Date: Jan. 15, 2009

(54) Aqueous fluid photoprotective compositions comprising tertiary-amide-terminated polyamide polymers

(75) Inventors: Anne-Laure Gaudry, Paris (FR); Martin Josso, Paris (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 12/216,890

(22) Filed: Jul. 11, 2008

Related U.S. Application Data
(60) Provisional application No. 60/959,923, filed on Jul. 18, 2007.

(30) Foreign Application Priority Data
Jul. 12, 2007 (FR) 0756456

(51) Int. Cl. A61K 9/12; A61K 8/33; A61P 17/18; B67D 5/54; B05B 9/03

(52) U.S. Cl. 424/45; 424/59; 222/373; 222/383.1; 239/337

(57) ABSTRACT

Topically applicable, fluid anti-sun/sunscreen compositions useful for protecting the skin and/or the hair against the damaging effects of ultraviolet radiation, contain:
(a) at least one photoprotective system for screening out UV radiation; and
(b) at least one tertiary-amide-terminated polyamide (ATPA) polymer, formulated into a topically applicable, cosmetically acceptable aqueous support therefor; the subject anti-sun/sunscreen compositions are especially provided as sprays, notably for the purpose of increasing the sun protection factor (SPF) and/or of reducing or even eliminating the fluffing effect thereof.

### CROSS-REFERENCE TO PRIORITY/PROVISIONAL APPLICATIONS

This application claims priority under 35 U.S.C. § 119 of FR 0756456, filed Jul. 12, 2007, and of U.S. Provisional Application No. 60/959,923, filed Jul. 18, 2007, each hereby expressly incorporated by reference and each assigned to the assignee hereof.

### BACKGROUND OF THE INVENTION

1. Technical Field of the Invention

The present invention relates to fluid compositions for protecting the skin and/or the hair against the damaging effects of ultraviolet radiation, which comprise, formulated into a cosmetically acceptable aqueous support:

(a) at least one photoprotective system capable of screening out UV radiation; and

(b) at least one tertiary-amide-terminated polyamide (ATPA) polymer.

The present invention more particularly relates to aqueous vaporizable fluid compositions, especially in the form of sprays, comprising a combination of:

(a) at least one photoprotective system capable of screening out UV radiation; and

(b) at least one tertiary-amide-terminated polyamide (ATPA) polymer.

This invention also relates to a device comprising at least (A) one reservoir containing at least one vaporizable fluid aqueous composition as defined above and (B) means for placing the said composition under pressure, in particular of the non-aerosol pump type (atomizer), or of the aerosol or aerosol pump type.

2. Description of Background and/or Related and/or Prior Art

It is known that light radiation with wavelengths of from 280 nm and 400 nm permits tanning of the human epidermis and that rays with wavelengths of from 280 and 320 nm, which are known as UV-B rays, cause skin burns and erythema that can harm the development of a natural tan; this UV-B radiation should thus be screened out.

It is also known that UV-A rays, with wavelengths of from 320 and 400 nm, which cause tanning of the skin, are liable to induce impairment thereof, especially in the case of sensitive skin or of skin that is continually exposed to solar radiation. UV-A rays in particular cause a loss of skin elasticity and the appearance of wrinkles, leading to premature aging. They promote the onset of the erythemal reaction or amplify this reaction in the case of certain individuals, and may even be the cause of phototoxic or photoallergic reactions. It is thus desirable also to screen out UV-A radiation.

Many photoprotective (UV-A and/or UV-B) cosmetic compositions for the skin have been proposed to date. Fluid formulations that afford for the users easy application to skin are most particularly sought.

These anti-sun/sunscreen fluid compositions are quite often in the form of an emulsion of oil-in-water type (i.e., a cosmetically acceptable support consisting of an aqueous dispersing continuous phase and of an oily dispersed discontinuous phase) that contains, in varying concentrations, one or more standard lipophilic and/or hydrophilic organic screening agents capable of selectively absorbing the harmful UV radiation, these screening agents (and the amounts thereof) being selected as a function of the desired sun protection factor, the sun protection factor (SPF) being expressed mathematically as the ratio of the dose of UV radiation required to reach the erythema-forming threshold with the UV-screening agent, to the dose of UV radiation required to reach the erythema-forming threshold without UV-screening agent.

Thus, there is an increasing need for fluid anti-sun products with a high protection factor. These high protection factors may be reached by incorporating more screening agents in high concentrations. This is not always achievable, despite the addition of large amounts of screening agents. Furthermore, such amounts may result in impairment of the comfort (tacky, coarse effect and/or greasy effect).

Anti-sun/sunscreen products in spray form are increasingly desired by consumers, on account of their ease of use and their cosmetic pleasantness.

To satisfy this objective, it has already been recommended, in EP-1,421,931, to employ spherical microparticles of porous silica. However, the sprays thus obtained have a tendency in certain cases to produce fluffing on the skin after application.

U.S. Pat. Nos. 6,469,131, 6,592,857, 6,552,160 and 6,268,466 disclose cosmetic compositions, especially makeup formulations and water-free sun protection products, in gel or solid composition form, comprising at least one tertiary-amide-terminated polyamide (ATPA) polymer, in which the said polymer is present as a gelling or structuring agent.

### SUMMARY OF THE INVENTION

After considerable research conducted in the field of photoprotection indicated above, it has now been discovered, surprisingly, that the formulation of a tertiary-amide-terminated polyamide (ATPA) polymer, into a fluid aqueous composition containing at least one system for screening out UV radiation, provides a fluid anti-sun/sunscreen composition with protection factors higher than those that may be obtained with the same photoprotective system alone, and in particular high-factor sprays, without the above drawbacks and disadvantages.

Thus, the present invention features novel fluid compositions for protecting the skin and/or the hair against ultraviolet radiation, which comprise, formulated into a cosmetically acceptable aqueous support:

(a) at least one photoprotective system capable of screening out UV radiation; and

(b) at least one tertiary-amide-terminated polyamide (ATPA) polymer.

According to the invention, the term “photoprotective system capable of screening out UV radiation” means any compound or any combination of compounds which, via mechanisms known per se of absorption and/or reflection and/or scattering of UV-A and/or UV-B radiation, makes it possible to prevent, or at least to limit, the contact of such radiation with a surface (skin or hair) onto which this or these compound(s) have been applied. In other words, these compounds may be UV-absorbing photoprotective organic screening agents or UV-scattering and/or UV-reflecting mineral pigments, and also mixtures thereof.

The term “cosmetically acceptable” means compatible with the skin and/or its integuments, which has a pleasant color, odor and feel and which does not cause any unacceptable discomfort (stinging, tautness or redness) liable to dissuade the consumer from using this composition.

The term “fluid composition” for the purposes of the invention means a composition which is not in a solid form and whose viscosity as measured using a Rheomat 180 viscometer at 25° C. at a rotation speed of 200 rpm after rotation for 10 minutes is less than or equal to 2 Pa·s.

The present invention also features the formulation of at least one tertiary-amide-terminated polyamide (ATPA) polymer into a fluid composition comprising, in a cosmetically acceptable aqueous support, at least one photoprotective system capable of screening out UV radiation, for the purpose of increasing the sun protection factor (SPF).

This invention also features the formulation of at least one tertiary-amide-terminated polyamide (ATPA) polymer in a fluid composition comprising, in a cosmetically acceptable aqueous support, at least one photoprotective system capable of screening out UV radiation, for the purpose of reducing or even eliminating the fluffing effect.

Other characteristics, aspects and advantages of the present invention will become apparent from the detailed description that follows.

### DETAILED DESCRIPTION OF BEST MODE AND SPECIFIC/PREFERRED EMBODIMENTS OF THE INVENTION

The tertiary-amide-terminated polyamide (ATPA) polymers in accordance with the present invention are preferably selected from among those conforming to the formula (1) below:

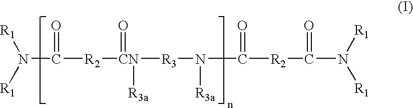

in which n is an integer greater than 0 which is a number of units such that the terminal amide groups containing R₁ constitute from 10% to 50% of the total number of amide groups in the formula (1);

the radicals R₁, which may be identical or different, are each a C₁-C₂₂ hydrocarbon group;

the radicals R₂, which may be identical or different, are each a C₂-C₄₂ hydrocarbon group, with the proviso that at least 50% of the radicals R₂ have 30 to 42 carbon atoms;

the radicals R₃, which may be identical or different, are each an organic group which has at least two carbon atoms in combination with hydrogen atoms and can additionally contain one or more oxygen or nitrogen atoms;

the radicals R₃ₐ, which may be identical or different, are each hydrogen, a C₁-C₁₀ alkyl radical, a direct bond with the radical R₃ or with another radical R₃ₐ so as to form, with the nitrogen atom bonded both to the radical R₃ and to the radical R₃ₐ, a heterocycle defined in part by R₃ₐ—N—R₃.

The tertiary-amide-terminated polyamides are obtained by reacting x equivalents of a dimer acid reactant of formula HOOC—R₂—COOH, y equivalents of a diamine reactant represented by the formula HN(R₃ₐ)—R₃—N(R₃ₐ)—H and z equivalents of a secondary monoamine reactant of formula R₁—NH—R₁, the equivalents x, y and z being other than 0.

In the formula (1), n is the number of units present in the formula (1) and is an integer greater than 0. According to the invention, n can be equal to 1, in which case the polyamide polymer contains the same number of terminal and non-terminal amide groups. The polyamides in accordance with the invention generally have a low molecular weight, such that n ranges preferably from 1 to 10 and more preferentially still from 1 to 5. In this case the polyamides of the invention may also be referred to as tertiary-amide-terminated oligoamides. From another standpoint, the terminal tertiary amide groups constitute preferably from 10% to 50%, more preferentially from 15% to 40% to more preferentially still from 20% to 35% of the total number of amides.

According to one preferred embodiment of the invention, the tertiary-amide-terminated polyamide polymer is a mixture of polymers of formula (1) with different values of n.

The tertiary-amide-terminated polyamide polymers preferably have a weight-average molecular weight of from 500 to 10,000, more preferentially from 1,000 and 5,000; this weight is measured by gel permeation chromatography using the polystyrene calibration standards.

The radicals R₁ are hydrocarbon groups and are preferably alkyl or alkenyl radicals containing at least one carbon atom, typically at least 4 carbon atoms: for example 8, 10, 12, 14, 16, 18, 20 or 22 carbon atoms. Alkyl groups are preferred, as are alkenyls having 1 to 3 and, more preferentially, one site of unsaturation. Preferentially, the radicals R₁ have a number of carbon atoms which is lower than or equal to 22. C₁₆-C₂₂ radicals are particularly preferred.

The appropriate groups R₁ are introduced into the molecule of formula (1) when a secondary monoamine R₁—NH—R₁ (R₁ having the same definition as that indicated above in the formula (1)) is used as a co-reactant in the preparation of the polyamides of the invention. Among the secondary monoamines which are available commercially, exemplary are those originating from various sources, for instance Witco Corporation (Greenwich, Conn.; http://www.witco.com); Akzo Nobel Chemicals, Surface Chemistry (Chicago, Ill.; http://www.akzonobelusa.com); and Aldrich (Milwaukee, Wis.; http://www.aldrich.sial.com).

Preferentially, use will be made of an amine or of a di-(C₁₄-C₁₈)alkylamine mixture.

The radicals R₂ are appropriately C₂-C₄₂ and preferably C₄-C₄₂ hydrocarbon groups, more preferentially still C₃₀-C₄₂ groups, and at least 50% of the groups R₂ are C₃₀-C₄₂. These groups are introduced during the preparation of the polyamide polymer from a polymerized fatty acid, also known as a dimer acid, having the formula HOOC—R₂—COOH(R₂ having the same definition as that indicated above in the formula (1)). The diacid is generally an organic molecule containing two carboxylic acid groups or equivalent reactive groups. Preferentially, the diacid is a polymerized fatty acid. The polymerized fatty acid is generally a mixture of structures, in which the dimers individually may be saturated, unsaturated, cyclic, acyclic, etc. A good description of the polymerization of fatty acids is indicated especially in the U.S. Pat. No. 3,157,681, in the work Naval Stores—Production Chemistry and Utilization, D. F. Zinkel and J. Russel (eds.). Among the dimer acids that are available commercially, exemplary are the product marketed under the trademark Unydime by Union Carbide Corporation (Wayne, N.J.), the dimer acid marketed under the trademark Empol by Henkel Corporation, Emery Oleochemicals Division (Cincinnati, Ohio), the dimer acid marketed under the trademark Pripol by Unichema North America (Chicago, Ill.) and the dimer acid marketed under the trademark Sylvadym by Arizona Chemical division of International Paper (Panama City, Fla.).

With particular preference, while the polyamides of the invention contain at least 50% of C₃₀-C₄₂ radicals R₂, the total number of C₃₀-C₄₂ groups R₂ is more preferentially at least 75% or even at least 90%.

The polyamides of the invention may also include groups R₂ having less than 30 carbon atoms, for example having 4 to 19 carbon atoms, more preferentially 4 to 8 carbon atoms. The carbon atoms may be arranged in linear, branched or cyclic formation, and an unsaturation may be present from two carbon atoms. R₂ may be aliphatic or aromatic. When they are present, these groups R₂ with a low carbon number are generally hydrocarbon groups (constituted solely of carbon and hydrogen atoms). These groups represent less than 50% of the total of the R₂ groups, and preferably from 5% to 35% of the total of the R₂ groups.

According to one particularly preferred embodiment of the invention, the group —(CO)—R₂—(CO)— will be a residue of the dimer of C₃₋₆ hydrogenated linoleic acid.

The group —N(R₃ₐ)—R₃—N(R₃ₐ)— is introduced into the molecule of formula (1) when a diamine HN(R₃ₐ)—R₃—N(R₃ₐ)H is used as a co-reactant in the preparation of the polyamide of the invention.

The group —N(R₃ₐ)—R₃—N(R₃ₐ)— is bonded to two carbonyl groups (C═O). According to one preferential embodiment of the invention, all of the groups R₃ₐ are hydrogens, so that the group R₃ is connected only to the two nitrogen atoms shown in the formula —N(R₃ₐ)—R₃—N(R₃ₐ)—. In this case the group R₃ contains at least two carbon atoms and optionally oxygen and/or nitrogen atoms, in addition to the hydrogen atoms which are necessary to fill the incomplete valencies of the carbon, nitrogen or oxygen atoms. According to one particular embodiment, R₃ is a hydrocarbon group having 2 to 36 carbon atoms, more preferentially 2 to 12 carbon atoms and more particularly still 2 to 8 carbon atoms. The carbon atoms may be arranged in linear, branched or cyclic formation and an unsaturation may be present from two carbon atoms. R₃ may be aliphatic or aromatic.

The groups R₃ may contain oxygen and/or nitrogen in addition to the carbon and hydrogen atoms. A typical oxygen-containing group R₃ is a polyoxyalkylene, i.e., a group having alternatively alkylene groups and oxygen atoms. The oxygenation in a group R₃ is preferably present in the form of an ether group. Among polyoxyalkylenes, exemplary are polyoxyethylenes, polyoxypropylenes and copolymers of ethylene oxide and propylene oxide (random, alternating or block). Oxygen-containing groups R₃ of this kind are introduced in the preparation of the polyamide of the invention by using Jeffamine diamines (Huntsman Chemical, Inc., Houston, Tex.). These materials are available in a wide range of molecular weights. When some of the radicals R₃ contain oxygen (at least 1%), preferably a minority of the groups R₃ possesses oxygen (less than 50%) and more preferentially less than 20% of the groups R₃ possess oxygen. The presence of oxygen in the group R₃ tends to lower the softening point of the polyamide.

When nitrogen atoms are present in the radical R₃, they are present in the form of secondary or tertiary amines. A typical nitrogen-containing group R₃ having secondary amine groups is a polyalkyleneamine, i.e., a group containing alternatively alkylene groups and amine groups, which may also be referred to as a polyalkylenepolyamine. The alkylene groups are generally lower alkylenes, for example methylene, ethylene (—CH₂—CH₂—), propylene, etc. One typical polyalkyleneamine may be represented by the formula —NH—(CH₂CH₂NH)ₘ—CH₂CH₂—NH— in which m is an integer from 1 to 5.

The nitrogen atoms in the group R₃ may alternatively (or additionally) be present in the form of tertiary nitrogen atoms, for example in a heterocycle of formula:

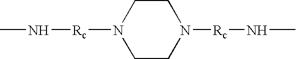

in which R_{c} is a C₁-C₃ alkylene radical.

In the nitrogen-containing groups R₃ as described above, the groups R₃ₐ are hydrogen. However, the groups R₃ₐ are not limited to hydrogen. They may also is a C₁-C₁₀ and more preferentially C₁-C₃ alkyl. Moreover, R₃ and R₃ₐ, or else two groups R₃ₐ, may together form a heterocycle, for example a piperazine of formula:

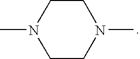

In this case, the two groups R₃ₐ may be seen as forming an ethylene bridge from the two nitrogen atoms, while R₃ is also an ethylene bridge.

More particularly, the group —N(R₃ₐ)—R₃—N(R₃ₐ)— will be a residue of ethylenediamine in which the radicals R₃ₐ are hydrogen and R₃ is —CH₂—CH₂—.

The diamines other than ethylenediamine will be designated in the text as co-diamines. When they are present, the co-diamines are used in small amounts relative to the ethylenediamine.

The tertiary-amide-terminated polyamide (ATPA) polymers in accordance with the invention are known per se and are described in U.S. Pat. Nos. 6,469,131, 6,592,857, 6,552,160 and 6,268,466. They may be prepared according to the process described in these documents.

Use will be made more particularly of the amide-terminated polyamide polymer ethylenediamide/hydrogenated dimer dilinoleate copolymer bis-di-C₁₄-C₁₈ alkyl amide (INCI name), which is a copolymer of hydrogenated linoleic diacid, ethylenediamine and di(C₁₄-C₁₈)alkylamine(s). This copolymer is especially marketed under the trademark Sylvaclear A200V by Arizona Chemical.

The amide-terminated polyamide polymer in accordance with the invention is present in the composition preferably in a maximum amount of 10% by weight, more preferentially from 0.1% to 10% by weight, even more preferentially from 0.5% to 5% to even more particularly from 1% to 3% relative to the total weight of the composition.

According to the invention, the photoprotective system may be constituted of one or more hydrophilic, lipophilic or insoluble organic screening agents and/or one or more mineral pigments. Preferentially, it will be constituted of at least one hydrophilic, lipophilic or insoluble organic UV-screening agent.

The organic UV-screening agents are selected especially from among cinnamic derivatives; anthranilates; salicylic derivatives; dibenzoylmethane derivatives; camphor derivatives; benzophenone derivatives; β,β-diphenylacrylate derivatives; triazine derivatives; benzotriazole derivatives; benzoalmalonate derivatives, especially those cited in U.S. Pat. No. 5,624,663; benzimidazole derivatives; imidazolines; bis-benzoazolyl derivatives as described in EP-669,323 and U.S. Pat. No. 2,463,264; p-aminobenzoic acid (PABA) derivatives; methylenebis(hydroxyphenylbenzotriazole) derivatives as described in U.S. Pat. Nos. 5,237,071, 5,166,355, GB-2,303,549, DE-197,26,184 and EP-893,119; benzoxazole derivatives as described in EP-0-832,642, EP-1,027,883, EP-1-300,137 and DE-101,62,844; screening polymers and screening silicones such as those described especially in WO 93/04665; α-alkylstyrene-derived dimers such as those described in DE-198,55,649; 4,4-diarylbutadienes such as those described in EP-0-967,200, DE-197,46,654, DE-197,55,649, EP-A-1-008,586, EP-1-133,980 and EP-133,981, merocyanin derivatives such as those described in WO 04/006878, WO 05/058269 and WO 06/032741; and mixtures thereof.

As examples of complementary organic photoprotective agents, representative are those denoted hereinbelow under their INCI name:

Cinnamic Derivatives:

Ethylhexyl methoxycinnamate marketed in particular under the trademark “Parsol MCX” by Hoffmann LaRoche,
Isopropyl methoxycinnamate,
Isoamyl methoxycinnamate marketed under the trademark “Neo Heliopan E 1000” by Haarmann and Reimer,
DEA methoxycinnamate,
Diisopropyl methylcinnamate,
Glyceryl ethylhexanoate dimethoxycinnamate.

Dibenzoylmethane Derivatives:

Butylmethoxydibenzoylmethane marketed especially under the trademark “Parsol 1789” by Hoffmann LaRoche,

### Isopropyldibenzoylmethane.

para-Aminobenzoic Acid Derivatives:

### PABA,

### Ethyl PABA,

Ethyl dihydroxypropyl PABA,
Ethylhexyl dimethyl PABA marketed in particular under the trademark “Escalol 507” by ISP,

### Glyceryl PABA,

PEG-25 PABA marketed under the trademark “Uvinul P25” by BASF.

Salicylic Derivatives:

Homosalate marketed under the trademark “Eusolex HMS” by Rona/EM Industries,
Ethylhexyl salicylate marketed under the trademark “Neo Heliopan OS” by Haarmann and Reimer,
Dipropylene glycol salicylate marketed under the trademark “Dipsal” by Scher,
TEA salicylate marketed under the trademark “Neo Heliopan TS” by Haarmann and Reimer.

β,β-Diphenylacrylate Derivatives:

Octocrylene marketed in particular under the trademark “Uvinul N539” by BASF,
Etocrylene marketed in particular under the trademark “Uvinul N35” by BASF.

Benzophenone Derivatives:

Benzophenone-1 marketed under the trademark “Uvinul 400” by BASF,
Benzophenone-2 marketed under the trademark “Uvinul D50” by BASF,
Benzophenone-3 or Oxybenzone marketed under the trademark “Uvinul M40” by BASF,
Benzophenone-4 marketed under the trademark “Uvinul MS40” by BASF,

### Benzophenone-5,

Benzophenone-6 marketed under the trademark “Helisorb 11” by Norquay,
Benzophenone-8 marketed under the trademark “Spectra-Sorb UV-24” by American Cyanamid,
Benzophenone-9 marketed under the trademark “Uvinul DS-49” by BASF,

### Benzophenone-12,

n-Hexyl 2-(4-diethylamino-2-hydroxybenzoyl)benzoate marketed under the trademark “Uvinal A+” or as a mixture with octylmethoxycinnamate under the trademark “Uvinul A+B” by BASF.

Benzylidenecamphor Derivatives:

3-Benzylidenecamphor manufactured under the trademark “Mexoryl SD” by Chimex,
4-Methylbenzylidenecamphor marketed under the trademark “Eusolex 6300” by Merck,
Benzylidenecamphorsulfonic acid manufactured under the trademark “Mexoryl SL” by Chimex,
Camphor benzalkonium methosulfate manufactured under the trademark “Mexoryl SO” by Chimex,
Terephthalylidenedicamphorsulfonic acid manufactured under the trademark “Mexoryl SX” by Chimex,
Polyacrylamidomethylbenzylidenecamphor manufactured under the trademark “Mexoryl SW” by Chimex.

Phenylbenzimidazole Derivatives:

Phenylbenzimidazolesulfonic acid marketed in particular under the trademark “Eusolex 232” by Merck,
Disodium phenyl dibenzimidazole tetrasulfonate marketed under the trademark “Neo Heliopan AP” by Haarmann and Reimer.

Phenylbenzotriazole Derivatives:

Drometrizole trisiloxane marketed under the trademark “Silatrizole” by Rhodia Chimie,
Methylenebis(benzotriazolyl)tetramethylbutylphenol marketed in solid form under the trademark “MIXXIM BB/100” by Fairmount Chemical, or in micronized form as an aqueous dispersion under the trademark “Tinosorb M” by Ciba Specialty Chemicals.

Triazine Derivatives:

bis-Ethylhexyloxyphenol Methoxyphenyl Triazine marketed under the trademark “Tinosorb S” by Ciba Geigy,
Ethylhexyltriazone marketed in particular under the trademark “Uvinul T150” by BASF,
Diethylhexylbutamidotriazone marketed under the trademark “Uvasorb HEB” by Sigma 3V,
2,4,6-tris(dineopentyl 4′-aminobenzalmalonate)s-triazine,
2,4,6-tris(diisobutyl 4′-aminobenzalmalonate)s-triazine,
2,4-bis(n-butyl 4′-aminobenzoate)-6-aminopropyltrisiloxane-s-triazine,
2,4-bis(dineopentyl 4′-aminobenzalmalonate)-6-(n-butyl 4′-aminobenzoate)-s-triazine,
symmetrical triazine screening agents described in U.S. Pat. No. 6,225,467, WO 2004/085412 (see compounds 6 and 9) or in “Symmetrical Triazine Derivatives” IP.COM Journal, IP.COM Inc., West Henrietta, N.Y., US (20 Sep. 2004), especially 2,4,6-tris(biphenyl-1,3,5-triazines (in particular 2,4,6-tris(biphenyl-4-yl)-1,3,5-triazine and 2,4,6-tris(terphenyl)-1,3,5-triazine, which is included in WO 06/035000, WO 06/034982, WO 06/034991, WO 06/035007, WO 2006/034992 and WO 2006/034985.

Anthranilic Derivatives:

Menthyl anthranilate marketed under the trademark “Neo Heliopan MA” by Haarmann and Reimer.

Imidazoline Derivatives:

Ethylhexyl dimethoxybenzylidenedioxoimidazolinepropionate.

Benzalmalonate Derivatives:

Polyorganosiloxane containing benzalmalonate functions, for instance Polysilicone-15, marketed under the trademark “Parsol SLX” by Hoffmann LaRoche.

4,4-Diarylbutadiene Derivatives:

1,1-Dicarboxy(2,2′-dimethylpropyl)-4,4-diphenylbutadiene.

Benzoxazole Derivatives:

2,4-bis[5-(1-dimethylpropyl)benzoxazol-2-yl(4-phenyl)imino]-6-(2-ethylhexyl)imino-1,3,5-triazine marketed under the trademark Uvasorb K2A by Sigma 3V,
and mixtures thereof.

The preferred organic screening agents are selected from among:

Ethylhexyl methoxycinnamate,

Ethylhexyl salicylate,

Homosalate,

Butylmethoxydibenzoylmethane,

Octocrylene,

Phenylbenzimidazolesulfonic acid,

Benzophenone-3,

Benzophenone-4,

Benzophenone-5,

n-Hexyl 2-(4-diethylamino-2-hydroxybenzoyl)benzoate,

4-Methylbenzylidenecamphor,

Terephthalylidenedicamphorsulfonic acid,

Disodium phenyldibenzimidazoletetrasulfonate,

Methylenebis-benzotriazolyl tetramethylbutylphenol,

Bis-ethylhexyloxyphenol methoxyphenyl triazine,

Ethylhexyl triazone,

Diethylhexyl butamido triazone,

2,4,6-Tris(dineopentyl 4′-aminobenzalmalonate)-s-triazine,

2,4,6-Tris(diisobutyl 4′-aminobenzalmalonate)-s-triazine,

2,4-Bis(n-butyl 4′-aminobenzoate)-6-aminopropyltrisiloxane-s-triazine,

2,4-Bis(dineopentyl 4′-aminobenzylmalonate)-6-(n-butyl 4′-aminobenzoate)-s-triazine,

2,4,6-Tris(biphenyl-4-yl)-1,3,5-triazine,

2,4,6-Tris(terphenyl)-1,3,5-triazine,

Drometrizole trisiloxane,

Polysilicone-15,

1,1-Dicarboxy(2,2′-dimethylpropyl)-4,4-diphenylbutadiene,

2,4-Bis[5-(1-dimethylpropyl)benzoxazol-2-yl(4-phenyl)imino]-6-(2-ethylhexyl)imino-1,3,5-triazine,

and mixtures thereof.

The inorganic screening agents used in accordance with the present invention are metal oxide pigments. More preferentially, the inorganic UV screening agents of the invention are metal oxide particles having an average elementary particle size of less than or equal to 500 nm, more preferentially from 5 nm to 500 nm, and more preferentially still from 10 nm to 100 nm, and preferentially from 15 to 50 nm.

They may be selected especially from titanium oxides, zinc oxides, iron oxides, zirconium oxides, cerium oxides or mixtures thereof.

Coated or uncoated metal oxide pigments of this kind are described more particularly in the EP-A-0-518,773. Commercial pigments that may be mentioned include the products marketed by Kemira, Tayca, Merck and Degussa.

The metal oxide pigments may be coated or uncoated.

The coated pigments are pigments that have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminum salts of fatty acids, metal alkoxides (of titanium or of aluminum), polyethylene, silicones, proteins (collagen, elastin), alkanolamines, silicon oxides, metal oxides or sodium hexametaphosphate.

The coated pigments are more particularly titanium oxides that have been coated:

with silica, such as the product “Sunveil” by Ikeda,

with silica and iron oxide, such as the product “Sunveil F” by Ikeda,

with silica and alumina, such as the products “Microtitanium Dioxide MT 500 SA” and “Microtitanium Dioxide MT 100 SA” by Tayca and “Tioveil” by Tioxide,

with alumina, such as the products “Tipaque TTO-55 (B)” and “Tipaque TTO-55 (A)” by Ishihara and “UVT 14/4” by Kemira,

with alumina and aluminum stearate, such as the products “Microtitanium Dioxide MT 100 T, MT 100 TX, MT 100 Z and MT-01 by Tayca, the products “Solaveil CT-10 W” and “Solaveil CT 100”, by Uniqema and the product “Eusolex T-AVO” by Merck,

with silica, alumina and alginic acid, such as the product “MT-100 AQ” by Tayca,

with alumina and aluminum laurate, such as the product “Microtitanium Dioxide MT 100 S” by Tayca,

with iron oxide and iron stearate, such as the product “Microtitanium Dioxide MT 100 F” by Tayca,

with zinc oxide and zinc stearate, such as the product “BR 351” by Tayca,

with silica and alumina and treated with a silicone, such as the products “Microtitanium Dioxide MT 600 SAS”, “Microtitanium Dioxide MT 500 SAS” or “Microtitanium Dioxide MT 100 SAS” by Tayca,

with silica, alumina and aluminum stearate and treated with a silicone, such as the product “STT-30-DS” by Titan Kogyo,

with silica and treated with a silicone, such as the product “UV-Titan X 195” by Kemira,

with alumina and treated with a silicone, such as the products “Tipaque TTO-55 (S)” by Ishihara or “UV Titan M 262” by Kemira,

with triethanolamine, such as the product “STT-65-S” by Titan Kogyo,

with stearic acid, such as the product “Tipaque TTO-55 (C)” by Ishihara,

with sodium hexametaphosphate, such as the product “Microtitanium Dioxide MT 150 W” by Tayca.

TiO₂ treated with octyltrimethylsilane marketed under the trademark “T 805” by Degussa Silices,

TiO₂ treated with a polydimethylsiloxane marketed under the trademark “70250 Cardre UF TiO2SI3” by Cardre,

anatase/rutile TiO₂ treated with a polydimethylhydrogenosiloxane marketed under the trademark “Microtitanium Dioxide USP Grade Hydrophobic” by Color Techniques.

The uncoated titanium oxide pigments are marketed, for example, by Tayca under the trademarks “Microtitanium Dioxide MT 500 B” or “Microtitanium Dioxide MT 600 B”, by Degussa under the trademark “P 25”, by Wacker under the trademark “Transparent titanium oxide PW”, by Miyoshi Kasei under the trademark “UFTR”, by Tomen under the trademark “ITS” and by Tioxide under the trademark “Tioveil AQ”.

The uncoated zinc oxide pigments are, for example:

those marketed under the trademark “Z-Cote” by Sunsmart;

those marketed under the trademark “Nanox” by Elementis;

those marketed under the trademark “Nanogard WCD 2025” by Nanophase Technologies.

The coated zinc oxide pigments are, for example:

those marketed under the trademark “Zinc Oxide CS-5” by Toshibi (ZnO coated with polymethylhydrogensiloxane);

those marketed under the trademark “Nanogard Zinc Oxide FN” by Nanophase Technologies (as a 40% dispersion in Finsolv TN, C₁₂-C₁₅ alkyl benzoate);

those marketed under the trademark “Daitopersion Zn-30” and “Daitopersion Zn-50” by Daito (dispersions in cyclopolymethylsiloxane/oxyethylenated polydimethylsiloxane, containing 30% or 50% of nanozinc oxides coated with silica and polymethylhydrogensiloxane);

those marketed under the trademark “NFD Ultrafine ZnO” by Daikin (ZnO coated with perfluoroalkyl phosphate and copolymer based on perfluoroalkylethyl as a dispersion in cyclopentasiloxane);

those marketed under the trademark “SPD-Z1” by Shin-Etsu (ZnO coated with silicone-grafted acrylic polymer, dispersed in cyclodimethylsiloxane);

those marketed under the trademark “Escalol Z100” by ISP (alumina-treated ZnO dispersed in an ethylhexyl methoxycinnamate/PVP-hexadecene/methicone copolymer mixture);

those marketed under the trademark “Fuji ZnO-SMS-10” by Fuji Pigment (ZnO coated with silica and polymethylsilsesquioxane);

those marketed under the trademark “Nanox Gel TN” by Elementis (ZnO dispersed at a concentration of 55% in C₁₂-C₁₅ alkyl benzoate with hydroxystearic acid polycondensate).

The uncoated cerium oxide pigments may be for example those marketed under the trademark “Colloidal Cerium Oxide” by Rhone-Poulenc.

The uncoated iron oxide pigments are marketed, for example, by Arnaud under the trademarks “Nanogard WCD 2002 (FE 45B)”, “Nanogard Iron FE 45 BL AQ”, “Nanogard FE 45R AQ” and “Nanogard WCD 2006 (FE 45R)” or by Mitsubishi under the trademark “TY-220”.

The coated iron oxide pigments are marketed, for example, by Arnaud under the trademarks “Nanogard WCD 2008 (FE 45B FN)”, “Nanogard WCD 2009 (FE 45B 556)”, “Nanogard FE 45 BL 345” and “Nanogard FE 45 BL” or by BASF under the trademark “Transparent Iron Oxide”.

Also exemplary are mixtures of metal oxides, especially of titanium dioxide and of cerium dioxide, including the silica-coated equal-weight mixture of titanium dioxide and of cerium dioxide, marketed by Ikeda under the trademark “Sunveil A”, and also the alumina-, silica- and silicone-coated mixture of titanium dioxide and of zinc dioxide, such as the product “M 261” marketed by Kemira, or the alumina-, silica- and glycerol-coated mixture of titanium dioxide and of zinc dioxide, such as the product “M 211” marketed by Kemira.

According to the invention, coated or uncoated titanium oxide pigments are particularly preferred.

The photoprotective system according to the invention is preferably present in the subject compositions in a content ranging from 0.1% to 40% by weight and in particular from 5% to 25% by weight relative to the total weight of the composition.

The aqueous compositions in accordance with the present invention may also comprise standard cosmetic adjuvants selected especially from fatty substances, organic solvents, ionic or nonionic, hydrophilic or lipophilic thickeners, demulcents, humectants, opacifiers, stabilizers, emollients, silicones, antifoams, fragrances, preservatives, anionic, cationic, nonionic, zwitterionic or amphoteric surfactants, fillers, polymers, propellants, acidifying or basifying agents or any other ingredient usually used in cosmetics and/or dermatology.

The fatty substances may consist of an oil or a wax other than the apolar waxes as defined above, or mixtures thereof. The term “oil” means a compound that is liquid at room temperature. The term “wax” means a compound that is solid or substantially solid at room temperature and whose melting point is generally greater than 35° C.

Oils that are exemplary include mineral oils (paraffin); plant oils (sweet almond oil, macadamia oil, grapeseed oil or jojoba oil); synthetic oils, for instance perhydrosqualene, fatty alcohols or fatty amides (for instance isopropyl lauroyl sarcosinate marketed under the trademark “Eldew SL-205” by Ajinomoto), fatty acids or fatty esters (for instance the C₁₂-C₁₅ alkyl benzoate marketed under the trademark “Finsolv TN” or “Witconol TN” by Witco, octyl palmitate, isopropyl lanolate and triglycerides, including capric/caprylic acid triglycerides, and dicaprylyl carbonate marketed under the trademark “Cetiol CC” by Cognis), oxyethylenated or oxypropylenated fatty esters and ethers; silicone oils (cyclomethicone and polydimethylsiloxanes, or PDMS) or fluoro oils, and polyalkylenes.

Waxy compounds that are exemplary include carnauba wax, beeswax, hydrogenated castor oil, polyethylene waxes and polymethylene waxes, for instance the product marketed under the trademark Cirebelle 303 by Sasol.

Among the organic solvents that are exemplary are lower alcohols and polyols. These polyols may be selected from glycols and glycol ethers, for instance ethylene glycol, propylene glycol, butylene glycol, dipropylene glycol or diethylene glycol.

Hydrophilic thickeners that are exemplary include carboxyvinyl polymers such as the Carbopol products (carbomers) and the Pemulen products (acrylate/C₁₀-C₃₀-alkylacrylate copolymer); polyacrylamides, for instance the crosslinked copolymers marketed under the trademarks Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by SEPPIC; 2-acrylamido-2-methylpropanesulfonic acid polymers and copolymers, which are optionally crosslinked and/or neutralized, for instance the poly(2-acrylamido-2-methylpropanesulfonic acid) marketed by Hoechst under the trademark “Hostacerin AMPS” (CTFA name: ammonium polyacryloyldimethyltaurate) or Simulgel 800 marketed by SEPPIC (CTFA name: sodium polyacryloyldimethyltaurate/polysorbate 80/sorbitan oleate); copolymers of 2-acrylamido-2-methylpropanesulfonic acid and of hydroxyethyl acrylate, for instance Simulgel NS and Sepinov EMT 10 marketed by SEPPIC; cellulose-based derivatives such as hydroxyethylcellulose; polysaccharides and especially gums such as xanthan gum; and mixtures thereof.

Lipophilic thickeners that are exemplary include synthetic polymers such as poly(C₁₀-C₃₀ alkyl acrylates) marketed under the trademark “Intelimer IPA 13-1” and “Intelimer IPA 13-6” by Landec, or modified clays such as hectorite and its derivatives, for instance the products marketed under the Bentone names.

As will be appreciated, one skilled in the art will take care to select the optional additional compound(s) mentioned above and/or the amounts thereof such that the advantageous properties intrinsically associated with the compositions in accordance with the invention are not, or are not substantially, adversely affected by the envisaged addition(s).

The compositions according to the invention may be prepared according to techniques that are well known to those skilled in the art. They may be in particular in the form of a simple or complex emulsion (O/W, W/O, O/W/O or W/O/W) such as a cream, a milk or a cream-gel; in the form of an aqueous gel; in the form of a lotion. They may optionally be packaged as an aerosol and may be in the form of a mousse or a spray.

The compositions according to the invention are preferably in the form of an oil-in-water or water-in-oil emulsion.

The emulsions generally contain at least one emulsifier selected from amphoteric, anionic, cationic and nonionic emulsifiers, which are used alone or as a mixture. The emulsifiers are appropriately selected according to the emulsion to be obtained (W/O or O/W). The emulsions may also contain stabilizers of other types, for instance fillers, gelling polymers or thickeners.

As emulsifying surfactants that may be used for the preparation of the W/O emulsions, examples thereof include sorbitan, glycerol or sugar alkyl esters or ethers; silicone surfactants, for instance dimethicone copolyols, such as the mixture of cyclomethicone and of dimethicone copolyol, marketed under the trademark “DC 5225 C” by Dow Corning, and alkyldimethicone copolyols such as laurylmethicone copolyol marketed under the trademark “Dow Corning 5200 Formulation Aid” by Dow Corning; cetyldimethicone copolyol, such as the product marketed under the trademark Abil EM 90R by Goldschmidt, and the mixture of cetyldimethicone copolyol, of polyglyceryl isostearate (4 mol) and of hexyl laurate, marketed under the trademark Abil WE 09 by Goldschmidt. One or more co-emulsifiers may also be added thereto, which may be selected advantageously from the group comprising polyol alkyl esters.

Polyol alkyl esters that are especially exemplary include polyethylene glycol esters, for instance PEG-30 dipolyhydroxystearate, such as the product marketed under the trademark Arlacel P135 by ICI.

Glycerol and/or sorbitan esters that are especially exemplary include polyglyceryl isostearate, such as the product marketed under the trademark Isolan GI 34 by Goldschmidt, sorbitan isostearate, such as the product marketed under the trademark Arlacel 987 by ICI, sorbitan glyceryl isostearate, such as the product marketed under the trademark Arlacel 986 by ICI, and mixtures thereof.

For the O/W emulsions, examples of emulsifiers include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters, for instance the mixture PEG-100 stearate/glyceryl stearate marketed, for example, by ICI under the trademark Arlacel 165; oxyalkylenated (oxyethylenated and/or oxypropylenated) fatty alkyl ethers; sugar esters, for instance sucrose stearate; fatty alkyl ethers of sugars, especially polyalkylglucosides (APG) such as decylglucoside and laurylglucoside marketed, for example, by Henkel under the respective names Plantaren 2000 and Plantaren 1200, cetostearyl glucoside optionally as a mixture with cetostearyl alcohol, marketed, for example, under the trademark Montanov 68 by SEPPIC, under the trademark Tegocare CG90 by Goldschmidt and under the trademark Emulgade KE3302 by Henkel, and also arachidyl glucoside, for example in the form of a mixture of arachidyl alcohol, behenyl alcohol and arachidyl glucoside, marketed under the trademark Montanov 202 by SEPPIC. According to one particular embodiment of the invention, the mixture of the alkylpolyglucoside as defined above with the corresponding fatty alcohol may be in the form of a self-emulsifying composition as described, for example, in WO-A-92/06778.

Among the other emulsion stabilizers, more particularly exemplary are isophthalic acid or sulfoisophthalic acid polymers, and in particular phthalate/sulfoisophthalate/glycol copolymers, for example the diethylene glycol/phthalate/isophthalate/1,4-cyclohexanedimethanol copolymer (INCI name: Polyester-5) marketed under the trademark “Eastman AQ Polymer” (AQ35S, AQ38S, AQ55S and AQ48 Ultra) by Eastman Chemical.

When it is an emulsion, the aqueous phase of this emulsion may comprise a nonionic vesicular dispersion prepared according to known processes (Bangham, Standish and Watkins, J. Mol. Biol., 13, 238 (1965), FR-2,315,991 and FR-2,416,008).

The compositions according to the invention find application in a large number of treatments, whether regime or regimen, especially cosmetic treatments, of the skin, the lips and the hair, including the scalp, especially for protecting and/or caring for the skin, the lips and/or the hair, and/or for making up the skin and/or the lips.

The present invention also features the use of the subject compositions for the production of cosmetic products for treating the skin, the lips, the nails, the hair, the eyelashes, the eyebrows and/or the scalp, especially care products, sun protection products and makeup products.

The cosmetic compositions according to the invention may be used, for example, as makeup products.

According to one preferred embodiment, the viscosity of the compositions as measured using a Rheomat 180 viscometer at 25° C. and at a rotation speed of 200 rpm after rotation for 10 minutes is from 0.01 to 2 and more preferentially from 0.01 to 0.5 Pa·s.

According to one particularly preferred embodiment, the compositions according to the invention are in vaporizable fluid form applied to the skin or the hair in the form of fine particles by means of pressurization devices.

According to the invention, the term “vaporizable composition” means any composition that can, under pressure in a suitable device, produce fine particles.

The present invention also features a pressurization device comprising at least (A) one reservoir containing at least one vaporizable fluid composition comprising, in a cosmetically acceptable aqueous support:

(a) at least one photoprotective system capable of screening out UV radiation as defined above;

(b) at least one tertiary-amide-terminated polyamide (ATPA) polymer as defined above; and

(B) means for placing the said composition under pressure.

The devices in accordance with the invention are well known to those skilled in the art and comprise non-aerosol pumps or “atomizers”, one- or two-compartment aerosol containers comprising a propellant, and also aerosol pumps using compressed air as propellant. These pumps are described in U.S. Pat. Nos. 4,077,441 and 4,850,517.

The compositions packaged in one-compartment aerosol form in accordance with the invention generally contain conventional propellants, for instance hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

The two-compartment aerosols are provided with a pocket in which the composition in accordance with the invention is present. The propellant is located in the can and to the exterior of the pocket. It remains inside the device during use and exerts pressure on the pocket. This propellant may be a liquefied gas such as the propellants used in one-compartment aerosols, but also a compressed gas, for instance air or nitrogen.

The compositions according to the invention may further comprise additional cosmetic and dermatological active agents.

It will be possible especially to choose the additional active agents from moisturizers, desquamating agents, agents for improving the barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, tensioning agents, lipid restructuring agents, slimming agents, agents for promoting the cutaneous microcirculation, calmatives and/or anti-irritants, sebo-regulating or anti-seborrhoeic agents, astringents, cicatrizing agents, anti-inflammatory agents, and anti-acne agents.

One skilled in the art will select the said active agent or agents as a function of the desired effect on the skin, hair, eyelashes, eyebrows or nails.

For caring for and/or making up skin which has aged, he or she will preferably select at least one active agent selected from moisturizers, desquamating agents, agents for improving the barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, lipid restructuring agents and agents promoting the cutaneous microcirculation for the area around the eyes.

It will be possible for the composition to further comprise at least one ingredient such as soft-focus effect fillers or agents which promote the natural coloring of the skin, for the purpose of complementing the biological effect of these actives or providing an immediate visual anti-age effect.

For caring for and/or making up greasy skin, one skilled in the art will preferably select at least one active agent selected from desquamating agents, sebo-regulating or anti-seborrhoeic agents and astringents.

It will be possible for the composition to further comprise at least one additional ingredient useful for complementing the biological effect of these actives or for providing an immediate visual effect; especially exemplary are matting agents, soft-focus effect fillers, fluorescers, agents for promoting the naturally pinkish coloration of the skin, and abrasive fillers or exfoliants.

Moisturizers or Humectants:

Moisturizers or humectants that are exemplary include glycerol and derivatives thereof, urea and derivatives thereof, especially Hydrovance® marketed by National Starch, lactic acid, hyaluronic acid, AHAs, BHAs, sodium pidolate, xylitol, serine, sodium lactate, ectoin and derivatives thereof, chitosan and derivatives thereof, collagen, plankton, an extract of Imperata cylindra marketed under the trademark Moist 24® by Sederma, acrylic acid homopolymers, for instance Lipidure-HM® from NOF Corporation, beta-glucan and in particular sodium carboxymethyl beta-glucan from Mibelle-AG-Biochemistry; a mixture of passionflower oil, apricot oil, corn oil and rice bran oil marketed by Nestlé under the trademark NutraLipids®; a C-glycoside derivative such as those described in WO 02/051 828 and in particular C-β-D-xylopyranoside-2-hydroxypropane in the form of a solution containing 30% by weight of active material in a water/propylene glycol mixture (60/40% by weight) such as the product marketed by Chimex under the trademark Mexoryl SBB®; an oil of musk rose marketed by Nestle; an extract of the microalga Prophyridium cruentum enriched with zinc, marketed by Vincience under the trademark Algualane Zinc®; spheres of collagen and of chondroitin sulfate of marine origin (Atelocollagen) marketed by Engelhard Lyon under the trademark Marine Filling Spheres; hyaluronic acid spheres such as those marketed by Engelhard Lyon; and arginine.

The moisturizer is preferably selected from urea and derivatives thereof, especially Hydrovance® marketed by National Starch, hyaluronic acid, AHAs, BHAs, acrylic acid homopolymers, for instance Lipidure-HM® from NOF Corporation, beta-glucan and in particular sodium carboxymethyl beta-glucan from Mibelle-AG-Biochemistry; a mixture of passionflower oil, apricot oil, corn oil and rice bran oil marketed by Nestle under the trademark NutraLipids®; a C-glycoside derivative such as those described in WO 02/051 828 and in particular C-β-D-xylopyranoside-2-hydroxypropane in the form of a solution containing 30% by weight of active material in a water/propylene glycol mixture (60/40% by weight) such as the product marketed by Chimex under the trademark Mexoryl SBB®; an oil of musk rose marketed by Nestle; an extract of the microalga Prophyridium cruentum enriched with zinc, marketed by Vincience under the trademark Algualane Zinc®; spheres of collagen and of chondroitin sulfate of marine origin (Atelocollagen) marketed by Engelhard Lyon under the trademark Marine Filling Spheres; hyaluronic acid spheres such as those marketed by Engelhard Lyon; and arginine.

Desquamating Agents:

The term “desquamating agent” means any compound capable of acting:

either directly on desquamation by promoting exfoliation, such as β-hydroxy acids (BHA), in particular salicylic acid and derivatives thereof (including 5-n-octanoylsalicylic acid, also known as capryloyl salicylic acid as the INCI name); α-hydroxy acids (AHA), such as glycolic acid, citric acid, lactic acid, tartaric acid, malic acid or mandelic acid; 8-hexadecene-1,16-dicarboxylic acid or 9-octadecenedioic acid; urea and derivatives thereof; gentisic acid and derivatives thereof; oligofucoses; cinnamic acid; Saphora japonica extract; resveratrol, and certain jasmonic acid derivatives;

or on the enzymes involved in the desquamation or degradation of corneodesmosomes, glycosidases, stratum corneum chymotryptic enzyme (SCCE) or other proteases (trypsin, chymotrypsin-like). Exemplary are aminosulfonic compounds and in particular 4-(2-hydroxyethyl)piperazine-1-propanesulfonic acid (HEPES); 2-oxothiazolidine-4-carboxylic acid (procysteine) and derivatives thereof; derivatives of α-amino acids of glycine type (as described in EP-0-852,949, and also sodium methyl glycine diacetate marketed by BASF under the trademark Trilon M); honey; sugar derivatives such as O-octanoyl-6-D-maltose and N-acetylglucosamine.

As other desquamating agents that may be included in the composition according to the invention, exemplary are:

oligofructoses, EDTA and derivatives thereof, laminaria extracts, o-linoleyl-6D-glucose, (3-hydroxy-2-pentylcyclopentyl)acetic acid, glycerol trilactate, O-octanyl-6′-D-maltose, S-carboxymethylcysteine, siliceous derivatives of salicylate such as those described in EP-0-796,861, oligofucases such as those described in EP-0-218,200, 5-acyl salicylic acid salts, actives with effects on transglutaminase, as in EP-0-899,330,

extract of the flowers of ficus Opuntia indica such as Exfolactive® from Silab,

8-hexadecene-1,16-dicarboxylic acid,

esters of glucose and of vitamin F, and

mixtures thereof.

Preferred desquamating agents include β-hydroxy acids such as 5-n-octanoyl salicylic acid; urea; glycolic acid, citric acid, lactic acid, tartaric acid, malic acid or mandelic acid; 4-(2-hydroxyethyl)piperazine-1-propanesulfonic acid (HEPES); extract of Saphora japonica; honey; N-acetyl glucosamine; sodium methyl glycine diacetate, and mixtures thereof.

Even more preferentially, a desquamating agent selected from 5-n-octanoyl salicylic acid; urea; 4-(2-hydroxyethyl)piperazine-1-propanesulfonic acid (HEPES); extract of Saphora japonica; honey; N-acetyl glucosamine; sodium methyl glycine diacetate, and mixtures thereof, will be included in the compositions of the invention.

Agents for Improving the Barrier Function:

As agents for improving the barrier function, especially exemplary are arginine, serine, an extract of Thermus thermophilus such as Venuceane® from Sederma, an extract of the rhizome of wild yam (Dioscorea villosa) such as Actigen Y® from Active Organics, plankton extracts, for instance Omega Plankton® from Secma, yeast extracts, for instance Relipidium® from Coletica, a chestnut extract such as Recoverine® from Silab, a cedar bud extract such as Gatuline Zen® from Gattefossé, sphingosines, for instance salicyloyl sphingosine marketed under the trademark Phytosphingosine® SLC by Degussa, a mixture of xylitol, polyxylityl glycoside and xylitan, for instance Aquaxyl® from SEPPIC, extracts of Solanacea plants, for instance Lipidessence® from Coletica, omega 3 unsaturated oils such as oils of musk rose; and mixtures thereof.

Especially exemplary are ceramides or derivatives thereof, in particular ceramides of type 2 (for instance N-oleoyldihydrosphingosine), of type 3 (for instance stearoyl-4-hydroxysphinganine, as the INCI name) and of type 5 (for instance N-2-hydroxypalmitoyidihydrosphingosine, having the INCI name: hydroxypalmitoyl sphinganine), sphingoid-based compounds, glycosphingolipids, phospholipids, cholesterol and derivatives thereof, phytosterols, essential fatty acids, diacylglycerol, 4-chromanone and chromone derivatives, petroleum jelly, lanolin, shea butter, cocoa butter, lanolin and PCA salts.

As preferred agents having a restructuring effect on the cutaneous barrier, exemplary are an extract of Thermus thermophilus, an extract of wild yam rhizome (Dioscorea villosa), a yeast extract, a chestnut extract, a cedar bud extract, arginine, serine, ceramides especially of type 3 and 5; and mixtures thereof.

Serine, arginine or a mixture thereof will preferably be employed.

Depigmenting Agents:

Depigmenting agents that are especially exemplary include vitamin C and derivatives thereof and especially vitamin CG, CP and 3-O ethyl vitamin C, alpha and beta arbutin, ferulic acid, lucinol and derivatives thereof, kojic acid, resorcinol and derivatives thereof, tranexamic acid and derivatives thereof, gentisic acid, homogentisate, methyl gentisate or homogentisate, dioic acid, calcium D-pantheteine sulfonate, lipoic acid, ellagic acid, vitamin B3, linoleic acid and derivatives thereof, ceramides and homologues thereof, plant derivatives, for instance camomile, bearberry, the aloe family (vera, ferox, bardensis), mulberry or skullcap; a kiwi fruit (Actinidia chinensis) juice marketed by Gattefossé, an extract of Paeonia suffruticosa root, such as the product marketed by Ichimaru Pharcos under the trademark Botanpi Liquid B®, an extract of brown sugar (Saccharum officinarum), such as the molasses extract marketed by Taiyo Kagaku under the trademark Molasses Liquid, without this list being exhaustive.

As preferred depigmenting agents, exemplary are vitamin C and its derivatives and especially vitamin CG, vitamin CP and 3-O-ethyl-vitamin C, alpha- and beta-arbutin, ferulic acid, kojic acid, resorcinol and its derivatives, calcium D-pantetheine sulfonate, lipoic acid, ellagic acid, vitamin B3, a kiwi fruit juice (Actinidia chinensis) marketed by Gattefossé, an extract of Paeonia suffruticosa root such as that marketed by Ichimaru Pharcos under the trademark BOTANPI LIQUID B®.

Antioxidants:

Especially exemplary are tocopherol and esters thereof, in particular tocopherol acetate; ascorbic acid and derivatives thereof, in particular magnesium ascorbyl phosphate and ascorbyl glucoside; ferulic acid; serine; ellagic acid, phloretin, polyphenols, tannins, tannic acid, epigallocatechins and natural extracts containing them, anthocyans, rosemary extracts, olive leaf extracts, for instance those by Silab, green tea extracts, resveratrol and derivatives thereof, ergothioneine, N-acetylcysteine, an extract of the brown alga Pelvetia canaliculata, for instance Pelvetiane® from Secma, chlorogenic acid, biotin, chelating agents, such as BHT and BHA, N,N′-bis(3,4,5-trimethoxybenzyl)ethylenediamine and salts thereof; idebenone, plant extracts, for instance Pronalen Bioprotect™ by Provital; coenzyme Q10, bioflavonoids, SODs, phytantriol, lignans, melatonin, pidolates, glutathione, caprylyl glycol, phloretin, Totarol™ or extract of Podocarpus totara containing Totarol (totara-8,11,13-trienol or 2-phenanthrenol, 4b,5,6,7,8,8a,9,10-octahydro-4b,8,8-trimethyl-1-(1-methylethyl)-; a jasmine extract such as the product marketed by Silab under the trademark Helisun®; hesperitin laurate such as Flavagrum PEG® by Engelhard Lyon; an extract of Paeonia suffruticosa root, such as the product marketed by Ichimaru Pharcos under the trademark Botanpi Liquid B® a lychee extract such as the lychee pericarp extract marketed by Cognis under the trademark Litchiderm LS 9704®, a pomegranate extract (Punica granatum), such as the product marketed by Draco Natural Products.

Other anti-aging agents that are exemplary include DHEA and derivatives thereof, boswellic acid, rosemary extracts, carotenoids (β-carotene, zeaxanthin and lutein), cysteic acid, copper derivatives and jasmonic acid.

Preferred antioxidants include ferulic acid; serine; phloretin, a pomegranate extract, biotin, chelating agents such as BHT, BHA, N,N′-bis(3,4,5-trimethoxybenzyl)ethylenediamine and salts thereof; caprylyl glycol, phloretin, Totarol™, a jasmine extract such as the product marketed by Silab under the trademark Helisun®; hesperitin laurate such as Flavagrum PEG® by Engelhard Lyon; an extract of Paeonia suffruticosa root, such as the product marketed by Ichimaru Pharcos under the trademark Botanpi Liquid B®.

Dermo-Relaxing or Dermo-Decontracting Agents:

Examples thereof include manganese gluconate and other salts, adenosine, alverine citrate and salts thereof, glycine, an extract of Iris pallida, a hexapeptide (Argeriline R from Lipotec) or sapogenins, for instance wild yam and the carbonyl amines described in EP-1-484,052. Examples of sapogenins include those described in WO 02/47650, in particular wild yam, the diosgenin extracted especially from Dioscorea opposita or any extract naturally containing or containing after treatment one or more sapogenins (wild yam rhizome, agave leaf, which contains hecogenin and tigogenin, extracts of Liliacea plants and more particularly yucca or smilax containing smilagenin and sarsapogenin, or sarsaparilla root) or Actigen Y by Actives Organics; or ginger.

Also exemplary are DMAE (dimethyl MEA), extracts of sea fennel, of rockrose, of helichrysum, of anise, of paracress, and an extract of Acmella oleracea, for instance Gatuline® from Gattefossé.

Preferred dermo-relaxing agents include adenosine, manganese gluconate, wild yam, sea fennel, glycine and alverine.

Anti-Glycation Agents:

The term “anti-glycation agent” means a compound that prevents and/or reduces the glycation of skin proteins, in particular dermal proteins such as collagen.

Anti-glycation agents that are exemplary include extracts of plants of the Ericacea family, such as an extract of blueberry (Vaccinium angustifolium or Vaccinium myrtillus), for example the product marketed under the trademark Blueberry Herbasol Extract PG by Cosmetochem, ergothioneine and derivatives thereof, hydroxystilbenes and derivatives thereof, such as resveratrol and 3,3′,5,5′-tetrahydroxystilbene (these anti-glycation agents are described in FR 2,802,425, FR 2,810,548, FR 2,796,278 and FR 2,802,420, respectively), dihydroxystilbenes and derivatives thereof, polypeptides of arginine and of lysine such as the product marketed under the trademark Amadorine® by Solabia, carcinine hydrochloride (marketed by Exsymol under the trademark Alistin®), an extract of Helianthus annuus, for instance Antiglyskin® from Silab, wine extracts such as the extract of powdered white wine on a maltodextrin support marketed under the trademark Vin blanc déshydraté 2F by Givaudan, thioctic acid (or alpha-lipoic acid), a mixture of extract of bearberry and of marine glycogen, for instance Aglycal LS 8777® from Laboratoires Serobiologiques, and an extract of black tea, for instance Kombuchka® from Sederma, and mixtures thereof.

Preferred anti-glycation agents include extracts of blueberry (Vaccinium myrtillus) and extract of black tea.

Agents for Stimulating the Synthesis of Dermal and/or Epidermal Macromolecules and/or for Preventing their Degradation:

Among the active agents for stimulating the dermal macromolecules or for preventing their degradation, exemplary are those acting:

either on collagen synthesis, such as extracts of Centella asiatica, asiaticosides and derivatives thereof; ascorbic acid or vitamin C and derivatives thereof; synthetic peptides such as iamin, biopeptide CL or palmitoyl oligopeptide marketed by Sederma; peptides extracted from plants, such as the soybean hydrolysate marketed by Coletica under the trademark Phytokine®; rice peptides such as Nutripeptide® from Silab, methylsilanol mannuronate such as Algisium C® marketed by Exsymol; plant hormones such as auxins and lignans; folic acid; and an extract of Medicago sativa (alfalfa) such as the product marketed by Silab under the trademark Vitanol®; a peptide extract of hazelnut such as the product marketed by Solabia under the trademark Nuteline C®; and arginine;

or on the inhibition of collagen degradation, in particular agents acting on the inhibition of metalloproteases (MMP) more particularly such as MMP 1, 2, 3 and 9. Mention may be made of: retinoids and derivatives, extracts of Medicago sativa such as Vitanol® from Silab, an extract of Aphanizomenon flos-aquae (Cyanophyceae) marketed under the trademark Lanablue® by Atrium Biotechnologies, oligopeptides and lipopeptides, lipoamino acids, the malt extract marketed by Coletica under the trademark Collalift®; blueberry or rosemary extracts; lycopene; isoflavones, derivatives thereof or plant extracts containing them, in particular extracts of soybean (marketed, for example, by Ichimaru Pharcos under the trademark Flavosterone SB®), of red clover, of flax or of kakkon; an extract of lychee such as the lychee pericarp extract marketed by Cognis under the trademark Litchiderm LS 9704®; Dipalmitoyl Hydroxyproline marketed by SEPPIC under the trademark Sepilift DPHP®: Baccharis genistelloide or Baccharine marketed by Silab, an extract of moring a such as Arganyl LS 9781® from Cognis; the sage extract described in FR-A-2 812 544 from the Labiatae family (Salvia officinalranges by Flacksmann), an extract of rhododendron, a blueberry extract, and an extract of Vaccinium myrtillus such as those described in FR-A-2 814 950;

or on the synthesis of molecules belonging to the elastin family (elastin and fibrillin), such as: retinol and derivatives, in particular retinol palmitate; the extract of Saccharomyces cerevisiae marketed by LSN under the trademark Cytovitin®; and the extract of the alga Macrocystis pyrifera marketed by Secma under the trademark Kelpadelie®; a peptide extract of hazelnut such as the product marketed by Solabia under the trademark Nuteline C®;

or on inhibition of elastin degradation, such as the peptide extract of seeds of Pisum sativum marketed by LSN under the trademark Parelastyl®; heparinoids; and the N-acylamino amide compounds described in WO 01/94381, such as {2-[acetyl(3-trifluoromethylphenyl)amino]-3-methylbutyrylamino}acetic acid, also known as N—[N-acetyl, N′-(3-trifluoromethyl)phenylvalyl]glycine, or N-acetyl-N-[3-(trifluoromethyl)phenyl]valyl glycine or acetyl trifluoromethyl phenyl valylglycine, or an ester thereof with a C₁-C₆ alcohol; an extract of rice peptides such as Colhibine from Pentapharm, or an extract of Phyllanthus emblica such as Emblica® from Rona;

or on the synthesis of glycosaminoglycans, such as the product of fermentation of milk with Lactobacillus vulgaris, marketed by Brooks under the trademark Biomin Yoghurt@; the extract of the brown alga Padina pavonica marketed by Alban Müller under the trademark HSP3®; the Saccharomyces cerevisiae extract available especially by Silab under the trademark Firmalift® or by LSN under the trademark Cytovitin®; an extract of Laminaria ochroleuca such as Laminaine® from Secma; essence of Mamaku from Lucas Meyer, and an extract of cress (Odraline® from Silab);

or on the synthesis of fibronectin, such as the extract of the zooplankton Salina marketed by Seporga under the trademark GP4G®; the yeast extract available especially by Alban Müller under the trademark Drieline®; and the palmitoyl pentapeptide marketed by Sederma under the trademark Matrixil®.

Among the active agents for stimulating epidermal macromolecules, such as fillagrin and keratins, especially exemplary are the extract of lupin marketed by Silab under the trademark Structurine®; the extract of Fagus sylvatica beech buds marketed by Gattefossé under the trademark Gatuline® RC; and the extract of the zooplankton Salina marketed by Seporga under the trademark GP4G®; the copper tripeptide from Procyte; a peptide extract of Voandzeia substerranea such as the product marketed by Laboratoires Se robiologiques under the trademark Filladyn LS 9397®.

Preferably, an active agent that stimulates the synthesis of dermal and/or epidermal macromolecules and/or that prevents their degradation, selected from agents for stimulating the synthesis of glycosaminoglycans, agents for inhibiting elastin degradation, agents for stimulating fibronectin synthesis, agents for stimulating the synthesis of epidermal macromolecules, and mixtures thereof, will be employed.

Even more preferentially, an active agent that stimulates the synthesis of the glycosaminoglycans, selected from an extract of the brown alga Padina pavonica, an extract of Saccharomyces cerevisiae, an extract of Laminaria ochroleuca, essence of Mamaku, and an extract of cress, and mixtures thereof, will even more preferentially be employed.

As preferred active agents for stimulating the synthesis of dermal and/or epidermal macromolecules and/or for preventing their degradation, exemplary are:

synthetic peptides such as iamin, the biopeptide CL or palmitoyloligopeptide marketed by Sederma; peptides extracted from plants, such as the soybean hydrolysate marketed by Coletica under the trademark Phytokine®; rice peptides such as Nutripeptide® from Silab, methylsilanol mannuronate such as Algisium C® marketed by Exsymol; folic acid; an extract of Medicago sativa (alfalfa), such as the product marketed by Silab under the trademark Vitanol®; a peptide extract of hazelnut, such as the product marketed by Solabia under the trademark Nuteline C®; arginine; an extract of Aphanizomenon flos-aquae (Cyanophyceae) marketed under the trademark Lanablue® by Atrium Biotechnologies, the malt extract marketed by Coletica under the trademark Collalift®, lycopene; an extract of lychee; an extract of moring a such as Arganyl LS 9781® from Cognis; an extract of Vaccinium myrtillus such as those described in FR-A-2 814 950; retinol and derivatives thereof, in particular retinyl palmitate; the extract of Saccharomyces cerevisiae marketed by LSN under the trademark Cytovitin®; a peptide extract of hazelnut such as the product marketed by Solabia under the trademark Nuteline C®; {2-[acetyl(3-trifluoromethylphenyl)amino]-3-methylbutyrylamino}acetic acid, also known as N—[N-acetyl, N′-(3-trifluoromethyl)phenylvalyl]glycine, or N-acetyl-N-[3-(trifluoromethyl)phenyl]valylglycine or acetyl trifluoromethyl phenyl valylglycine, or an ester thereof with a C₁-C₆ alcohol; an extract of rice peptides such as Colhibin® from Pentapharm, or an extract of Phyllanthus emblica such as Emblica® from Rona; the extract of the brown alga Padina pavonica marketed by Alban Müller under the trademark HSP3®; the extract of Saccharomyces cerevisiae available especially by Silab under the trademark Firmalift® or by LSN under the trademark Cytovitin®; an extract of Laminaria ochroleuca such as Laminaine® from Secma; the essence of Mamaku from Lucas Meyer, the extract of lupin marketed by Silab under the trademark Structurine®; the extract of Fagus sylvatica beech buds marketed by Gattefossé under the trademark Gatuline® RC.

Agents for Stimulating Fibroblast or Keratinocyte Proliferation and/or Keratinocyte Differentiation:

The agents for stimulating fibroblast proliferation that may be included in the compositions according to the invention may be selected, for example, from plant proteins or polypeptides, extracted especially from soybean (for example a soybean extract marketed by LSN under the trademark Eleseryl SH-VEG 8® or marketed by Silab under the trademark Raffermine®); an extract of hydrolyzed soybean proteins such as Ridulisse® from Silab; and plant hormones such as gibberellins and cytokinins; a peptide extract of hazelnut such as the product marketed by Solabia under the trademark Nuteline C®.

Preferably, an agent that promotes keratinocyte proliferation and/or differentiation will be employed.

The agents for stimulating keratinocyte proliferation that may be included in the compositions according to the invention especially comprise adenosine; phloroglucinol, the extract of Hydrangea macrophylla leaves, for instance Amacha Liquid E® from Ichimaru Pharcos, a yeast extract such as Stimoderm® from CLR; the extract of Larrea divaricata such as Capislow® from Sederma, mixtures of extracts of papaya, of olive leaves and of lemon, such as Xyleine® from Vincience, the extract of Hydrangea macrophylla leaves, for instance Amacha Liquid E® from Ichimaru Pharcos, retinol and esters thereof, including retinyl palmitate, phloroglucinol, the nut cake extracts marketed by the Gattefossé and the extracts of Solanum tuberosum such as Dermolectine® marketed by Sederma.

Among the agents for stimulating keratinocyte differentiation are, for example, minerals such as calcium; sea fennel, a peptide extract of lupin, such as the product marketed by Silab under the trademark Structurine®; sodium beta-sitosteryl sulfate, such as the product marketed by Seporga under the trademark Phytocohesine®; and a water-soluble extract of corn, such as the product marketed by Solabia under the trademark Phytovityl®; a peptide extract of Voandzeia substerranea such as the product marketed by Laboratoires Serobiologiques under the trademark Filladyn LS 9397®; and lignans such as secoisolariciresinol, and retinol and esters thereof, including retinyl palmitate.

As agents for stimulating keratinocyte proliferation and/or differentiation, exemplary are the oestrogens such as oestradiol and homologues; cytokines.

As preferred active agents for stimulating fibroblast or keratinocyte proliferation and/or keratinocyte differentiation, exemplary are plant proteins or polypeptides, extracted especially from soybean (for example a soybean extract marketed by LSN under the trademark Eleseryl SH-VEG 8® or marketed by Silab under the trademark Raffermine®); an extract of hydrolyzed soybean proteins such as Ridulisse® from Silab; a peptide extract of hazelnut such as the product marketed by Solabia under the trademark Nuteline C®; adenosine; phloroglucinol, a yeast extract such as Stimoderm® from CLR; a peptide extract of lupin such as the product marketed by Silab under the trademark Structurine®; a water-soluble corn extract, such as the product marketed by Solabia under the trademark Phytovityl®; a peptide extract of Voandzeia substerranea, such as the product marketed by Laboratoires Serobiologiques under the trademark Filladyn LS 9397®; retinol and esters thereof, including retinyl palmitate.

Agents for Promoting the Maturation of the Horny Envelope:

Agents that participate in the maturation of the horny envelope, which becomes impaired with age and induces a decrease in transglutaminase activity, may be included in the compositions of the invention. Examples are urea and derivatives thereof and in particular Hydrovance® from National Starch and the other active agents mentioned in L'Oréal FR 2 877 220 (unpublished).

NO-Synthase Inhibitors:

The agent with an inhibitory action on NO synthase may be selected from OPCs (procyannidol oligomers); plant extracts of the species Vitis vinifera marketed especially by Euromed under the trademark “Leucocyanidines de raisins extra”, or by Indena under the trademark Leucoselect®, or finally by Hansen under the trademark “Extrait de marc de raisin”; plant extracts of the species Olea europaea preferably obtained from olive tree leaves and marketed especially by Vinyals in the form of a dry extract, or by Biologia & Technologia under the trademark Eurol® BT; and plant extracts of the species Gingko biloba, preferably a dry aqueous extract of this plant marketed by Beaufour under the trademark “Ginkgo biloba extrait standard”, and mixtures thereof.

Peripheral Benzodiazepine Receptor (PBR) Antagonists:

Exemplary are 1-(2-chlorophenyl)-N-(1-methylpropyl)-3-isoquinoline carboxamide; the compounds described in WO 03/030 937 and WO 03/068 753, pyridazino[4,5-b]indole-1-acetamide derivatives of general formula (VII) as described in WO 00/44384.

Agents for Increasing the Activity of the Sebaceous Glands:

Exemplary are methyl dehydrojasmonate, hecogenin, hedione and O-linoleyl-6D-glucose, and mixtures thereof.

Agents for Stimulating the Energy Metabolism of Cells:

The active agent for stimulating the energy metabolism of cells may be selected, for example, from biotin, an extract of Saccharomyces cerevisiae such as Phosphovital® from Sederma, the mixture of sodium, manganese, zinc and magnesium salts of pyrrolidonecarboxylic acid, for instance Physiogenyl® from Solabia, a mixture of zinc, copper and magnesium gluconate, such as Sepitonic M3® from SEPPIC, and mixtures thereof; a beta-glucan derived from Saccharomyces cerevisiae, such as the product marketed by Mibelle AG Biochemistry.

Tensioning Agents:

The term “tensioning agent” according to the invention means compounds having a tensioning effect, i.e., being able to make the skin taut.

According to the invention, the term “tensioning agent” generally means any compound that is soluble or dispersible in water at a temperature ranging from 25° C. to 50° C. at a concentration of 7% by weight in water or at the maximum concentration at which a medium of uniform appearance is formed and producing at this concentration of 7% or at this maximum concentration in water a shrinkage of more than 15% in the test described below.

The maximum concentration at which a medium of uniform appearance forms is determined to within ±10% to preferably to within ±5%.

The expression “medium of uniform appearance” means a medium that does not contain any aggregates that are visible to the naked eye.

For the determination of the said maximum concentration, the tensioning agent is gradually added to the water with deflocculating stirring at a temperature ranging from 25° C. to 50° C., and the mixture is then stirred for one hour. The mixture thus prepared is then examined after 24 hours to see if it is of uniform appearance (absence of aggregates visible to the naked eye).

The tensioning effect may be characterized by an in vitro shrinkage test.

A homogeneous mixture of the tensioning agent in water, at a concentration of 7% by weight or at the maximum concentration described above, is prepared beforehand and as described previously.

30 μl of the homogeneous mixture are placed on a rectangular sample (10×40 mm, thus having an initial width L₀ of 10 mm) of elastomer with a modulus of elasticity of 20 MPa and a thickness of 100 μm.

After drying for 3 hours at 22±3° C. and 40±10% relative humidity RH, the elastomer sample has a shrunken width, noted L₃ₕ, due to the tension exerted by the applied tensioning agent.

The tensioning effect (TE) of the said polymer is then quantified in the following manner:

‘TE’=(L₀−L₃ₕ/L₀)×100 as % with L₀=initial width 10 mm and L₃ₕ=width after 3 hours of drying

The tensioning agent may be selected from:

plant or animal proteins and hydrolysates thereof;

polysaccharides of natural origin;

mixed silicates;

colloidal particles of mineral fillers;

synthetic polymers;

and mixtures thereof.

One skilled in this art will know how to choose, from the chemical categories listed above, the materials corresponding to the tensioning test as described above.

Especially exemplary are:

(a) plant proteins and protein hydrolysates, in particular of corn, rye, wheat, buckwheat, sesame, spelt, pea, bean, lentil, soybean and lupin,

(b) polysaccharides of natural origin, especially (a) polyholosides, for example (i) in the form of starch derived especially from rice, corn, potato, cassaya, pea, wheat, oat, etc. or (ii) in the form of carrageenans, alginates, agars, gellans, cellulose polymers and pectins, advantageously as an aqueous dispersion of gel microparticles, and (b) latices consisting of shellac resin, sandarac gum, dammar resins, elemi gums, copal resins, cellulose derivatives, and mixtures thereof,

(c) mixed silicates, especially phyllosilicates and in particular Laponites,

(d) colloidal particles of mineral fillers with a number-average diameter of from 0.1 and 100 nm and preferably from 3 and 30 nm, and selected, for example, from: silica, silica-alumina composites, cerium oxide, zirconium oxide, alumina, calcium carbonate, barium sulfate, calcium sulfate, zinc oxide and titanium dioxide. As silica-alumina composite colloidal particles that may be included in the compositions according to the invention, examples include those marketed by Grace under the trademarks Ludox AM, Ludox AM-X 6021, Ludox HSA and Ludox TMA,

(e) synthetic polymers, such as polyurethane latices or acrylic-silicone latices, in particular those described in EP-1-038,519, such as a polydimethylsiloxane grafted with propylthio(polymethyl acrylate), propylthio(polymethyl methacrylate) and propylthio(polymethacrylic acid), or, alternatively, a polydimethylsiloxane grafted with propylthio(polyisobutyl methacrylate) and propylthio(polymethacrylic acid). Such grafted silicone polymers are especially marketed by 3M under the trademarks VS 80, VS 70 and LO21.

The tensioning agent will be present in the composition in an amount that is effective for obtaining the desired biological effect according to the invention.

By way of example, the tensioning agent may be included in the compositions according to the invention in a content ranging from 0.01% to 30% by weight of active material and preferably from 1% to 30% by weight of active material relative to the total weight of the composition.

The term “active material” is intended to exclude the medium in which the tensioning agent may be dissolved or dispersed in its commercial form, for example in the case of dispersions of colloidal particles.

It is also possible, especially for complementing and/or potentializing the effect of tensioning agents, to employ agents which increase the expression of mechanoreceptors, such as agents which increase the expression of integrins.

An example is a rye seed extract, such as that marketed by Silab under the trademark Coheliss®.

Fat Restructuring Agents:

“Fat restructuring agents” are, according to the invention, agents which are capable of stimulating lipogenesis and promoting adipocyte differentiation, thereby making it possible to prevent or slow down the wasting of the fats contained in the support tissues of the skin, as is also called ‘wasting of the fat structure of the skin’.

‘Fat structure of the skin’ means the network of fat cells which form the volumes over which the facial skin rests and moulds itself to.

These agents are useful for lessening the loss of skin density and/or the wasting of the fat structure of the skin, more particularly on the cheeks and in the area around the eye, and/or for preventing the collapse and/or hollowing of the volumes of the face, the loss of consistency of the skin and/or its maintenance, more particularly on the cheeks and in the area around the eye, and/or for improving the underlying volumes of the skin of the face and/or neck, more particularly on the cheeks, of the oval of the face and of the area around the eye, and/or for improving the density, springiness and maintenance of the skin, more particularly on the cheeks, of the oval of the face and of the area around the eye, and/or of remodeling the features of the face, more particularly the oval of the face.

Examples of fat restructuring agents include, especially, a black tea extract, such as the extract of fermented black tea that is marketed by Sederma under the trademark Kombuchka®, and an extract of Artemisia abrotanum, such as that marketed by Silab under the trademark Pulpactyl®.

Slimming Agents:

Slimming (lipolytic) agents that are especially exemplary include caffeine, theophylline and its derivatives, theobromine, sericosine, asiatic acid, acefylline, aminophylline, chloroethyltheophylline, diprofylline, diniprophylline, etamiphylline and its derivatives, etofylline and proxyphylline; extracts of tea, of coffee, of guarana, of mate, of cola (Cola nitida) and especially the dry extract of guarana fruit (Paulina sorbilis) containing 8% to 10% caffeine; extracts of climbing ivy (Hedera helix), of arnica (Amica montana L), of rosemary (Rosmarinus officinalis N), of marigold (Calendula officinalis), of sage (Salvia officinalis L), of ginseng (Panax ginseng), of St.-John's wort (Hypericum perforatum), of butcher's-broom (Ruscus aculeatus L), of meadowsweet (Filipendula ulmaria L), of orthosiphon (Orthosiphon stamincus Benth), of birch (Betula alba), of pumpwood and of argan tree, extracts of ginkgo biloba, extracts of horsetail, extracts of escin, extracts of cangzhu, extracts of Chrysanthellum indicum, extracts of diosgenin-rich Dioscorea plants or pure diosgenin or hecogenin and derivatives thereof, extracts of Ballota, extracts of Guioa, of Davallia, of Terminalia, of Barringtonia, of Trema or of Antirobia, the extract of bitter orange pips; an extract of husks of cocoa beans (Theobroma cacao) such as the product marketed by Solabia under the trademark Caobromine®.

Agents for Promoting the Cutaneous Microcirculation:

The active agent acting on the cutaneous microcirculation may be used for preventing dulling of the complexion and/or for improving the appearance of the area around the eye, especially for reducing shadows. It may be selected, for example, from an extract of maritime pine bark, for instance Pycnogenol® from Biolandes, manganese gluconate (Givobio GMn® from SEPPIC), an extract of Ammi visnaga such as Visnadine from Indena, extract of lupin (Eclaline® from Silab), the protein coupling of hydrolyzed wheat/palmitic acid with palmitic acid, such as Epaline 100 from Laboratoires Carilène, the extract of bitter orange blossom (Remoduline® from Silab), vitamin P and derivatives thereof, for instance methyl-4 esculetol sodium monoethanoate marketed under the trademark Permethol® by Sephytal, extracts of Ruscus, of common horse chestnut, of ivy, of ginseng and of melilot, caffeine, nicotinate and derivatives thereof, lysine and derivatives thereof, for instance Asparlyne® from Solabia, an extract of black tea such as Kombuchka from Sederma; rutin salts; an extract of the alga Corallina officinalis, such as the product marketed by Codif; and mixtures thereof.

As preferred agents for promoting the cutaneous microcirculation, exemplary are caffeine, an extract of bitter orange blossom, an extract of black tea, rutin salts and an extract of the alga Corallina officinalis.

Calmatives or Anti-Irritants:

The term “calmative” means a compound that reduces the sensation of stinging, itching or tautness of the skin.

As calmatives that may be included in the compositions according to the invention, exemplary are:

procyannidol oligomers, vitamins E, C, B5 and B3, caffeine and derivatives thereof, pentacylic triterpenes and plant extracts containing them, β-glycyrrhetinic acid and salts or derivatives thereof (stearyl glycyrrhetate, 3-stearoyloxyglycyrrhetic acid or glycyrrhetinic acid monoglucuronide) and also plants containing them (e.g., Glycyrrhiza glabra), oleanolic acid and salts thereof, ursolic acid and salts thereof, boswellic acid and salts thereof, betulinic acid and salts thereof, an extract of Paeonia suffruticosa and/or lactiflora, an extract of Laminaria saccharina, extracts of Centella asiatica, Canola oil, bisabolol, the phosphoric diester of vitamin E and C, for instance Sepivital EPC® from SEPPIC, camomile extracts, allantoin, omega-3 unsaturated oils such as musk rose oil, blackcurrant oil, Ecchium oil, fish oil or beauty-leaf oil, plankton extracts, capryloyl glycine, a mixture of water lily blossom extract and of palmitoylproline, such as the product marketed under the trademark Seppicalm VG® by SEPPIC, an extract of Boswellia serrata, an extract of Centipeda cunninghami, such as the product marketed under the trademark Cehami PF® by TRI-K Industries, an extract of sunflower seeds, in particular Hélioxine® from Silab, an extract of Linum usitatissimum seeds, for instance Sensiline® from Silab, tocotrienols, piperonal, an extract of Epilobium angustifolium, such as the product marketed under the trademark Canadian Willowherb Extract by Fytokem Products, Aloe vera, phytosterols, cornflower water, rose water, an extract of mint, in particular of mint leaves, for instance Calmiskin® from Silab, anise derivatives, filamentous bacteria, for instance Vitreoscilla filiformis as described in EP-761,204 and marketed by Chimex under the trademark Mexoryl SBG®, an extract of rose petals, for instance Rose Flower Herbasol® extract by Cosmetochem, shea butter, a mixture of the waxy fraction of barley seeds obtained by supercritical CO₂, of shea butter and of argan oil, for instance Stimu-tex AS® from Pentapharm, alkaline-earth metal salts, especially of strontium, a fermented extract of Alteromonas marketed under the trademark Abyssine® by Atrium Biotechnologies; spring water from the Vichy basin, such as waters originating from the Celestins, Chomel, Grande-Grille, Hopital, Lucas and Parc sources, and preferably water from the Lucas source; an extract of Eperua falcata bark, such as the product marketed by Cognis under the trademark Eperuline®; an extract of Paeonia suffruticosa root, such as the product marketed by Ichimaru Pharcos under the trademark Botanpi Liquid B®; and mixtures thereof.

Preferred calmatives according to the invention include:

β-glycyrrhetinic acid and salts or derivatives thereof (stearyl glycyrrhetate, 3-stearoyloxyglycyrrhetic acid or glycyrrhetinic acid monoglucuronide) and also plants containing them (e.g. Glycyrrhiza glabra); ursolic acid and salts thereof; extracts of Centella asiatica, Canola oil, bisabolol; camomile extracts, allantoin; a mixture of extract of water lily blossom and of palmitoylproline, such as the product marketed under the trademark Seppicalm VG® by SEPPIC; Aloe vera, rose water, extract of mint, in particular of mint leaves, such as Calmiskin® from Silab, filamentous bacteria such as Vitreoscilla filiformis as described in EP-761,204 and marketed by Chimex under the trademark Mexoryl SBG®, an extract of rose petals such as Rose Flower Herbasol® extract by Cosmetochem, shea butter, a fermented extract of Alteromonas marketed under the trademark Abyssine® by Atrium Biotechnologies; spring water from the Vichy basin, such as waters originating from the Célestins, Chomel, Grande-Grille, Hopital, Lucas and Parc sources, and preferably water from the Lucas source; an extract of Eperua falcata bark, such as the product marketed by Cognis under the trademark Eperuline®; an extract of Paeonia suffruticosa root, such as the product marketed by Ichimaru Pharcos under the trademark Botanpi Liquid B®; and mixtures thereof.

Sebo-Regulating or Anti-Seborrhoeic Agents:

The term “sebo-regulating or anti-seborrhoeic agents” especially means agents capable of regulating the activity of the sebaceous glands.

Especially exemplary are:

retinoic acid, benzoyl peroxide, sulfur, vitamin B6 (or pyridoxine), selenium chloride and sea fennel;

mixtures of extract of cinnamon, of tea and of octanoylglycine such as Sepicontrol A5 TEA® from SEPPIC;

the mixture of cinnamon, sarcosine and octanoylglycine marketed especially by SEPPIC under the trademark Sepicontrol A5®;

zinc salts such as zinc gluconate, zinc pyrrolidonecarboxylate (or zinc pidolate), zinc lactate, zinc aspartate, zinc carboxylate, zinc salicylate and zinc cysteate;

copper derivatives and in particular copper pidolate such as Cuivridone® from Solabia;

extracts of plants of the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all marketed, for example, by Maruzen;

extracts of meadowsweet (Spiraea ulmaria), such as the product marketed under the trademark Sebonormine® by Silab;

extracts of the alga Laminaria saccharina, such as the product marketed under the trademark Phlorogine® by Biotechmarine;

mixtures of extracts of salad burnet root (Sanguisorba officinalis/Poterium officinale), of ginger rhizomes (Zingiber officinalis) and of cinnamon bark (Cinnamomum cassia), such as the product marketed under the trademark Sebustop® by Solabia;

linseed extracts, such as the product marketed under the trademark Linumine® by Lucas Meyer;

Phellodendron extracts, such as those marketed under the trademark Phellodendron extract BG by Maruzen or Oubaku liquid B by Ichimaru Pharcos;

mixtures of argan oil, of Serenoa serrulata (saw palmetto) extract and of sesame seed extract, such as the product marketed under the trademark Regu SEB® by Pentapharm;

mixtures of extracts of willowherb, of Terminalia chebula, of nasturtium and of bioavailable zinc (microalgae), such as the product marketed under the trademark Seborilys® by Green Tech;

extracts of Pygeum afrianum, such as the product marketed under the trademark Pygeum afrianum sterolic lipid extract by Euromed;

extracts of Serenoa serrulata, such as the products marketed under the trademark Viapure Sabal by Actives International or those marketed by Euromed;

mixtures of extracts of plantain, of Berberis aquifolium and of sodium salicylate, such as the product marketed under the trademark Seboclear® by Rahn;

clove extract, such as the product marketed under the trademark Clove extract powder by Maruzen;

argan oil, such as the product marketed under the trademark Lipofructyl® by Laboratoires Serobiologiques;

lactic protein filtrates, such as the product marketed under the trademark Normaseb® by Sederma;

extracts of the alga Laminaria, such as the product marketed under the trademark Laminarghane® by Biotechmarine;

oligosaccharides of the alga Laminaria digitata, such as the product marketed under the trademark Phycosaccharide AC by Codif;

sugar cane extracts, such as the product marketed under the trademark Policosanol® by Sabinsa;

sulfonated shale oil, such as the product marketed under the trademark Ichthyol Pale® by Ichthyol;

European meadowsweet (Spiraea ulmaria) extracts, such as the product marketed under the trademark Cytobiol® Ulmaire by Libiol;

sebacic acid, especially marketed in the form of a sodium polyacrylate gel under the trademark Sebosoft® by Sederma;

glucomannans extracted from konjac tuber and modified with alkylsulfonate chains, such as the product marketed under the trademark Biopol Beta by Arch Chemical;

extracts of Sophora angustifolia, such as those marketed under the trademark Sophora powder or Sophora extract by Bioland;

extracts of Cinchona succirubra bark, such as the product marketed under the trademark Red Bark HS by Alban Muller;

extracts of Quillaja saponaria, such as the product marketed under the trademark Panama wood HS by Alban Muller;

glycine grafted onto an undecylenic chain, such as the product marketed under the trademark Lipacide UG OR by SEPPIC;

the mixture of oleanolic acid and of nordihydroguaiaretic acid, such as the product marketed in the form of a gel under the trademark AC.Net by Sederma;

phthalimidoperoxyhexanoic acid;

tri(C₁₂-C₁₃)alkyl citrate marketed under the trademark Cosmacol® ECI by Sasol; tri(C₁₄-C₁₅)alkyl citrate marketed under the trademark Cosmacol® ECL by Sasol;

10-hydroxydecanoic acid, and especially mixtures of 10-hydroxydecanoic acid, of sebacic acid and of 1,10-decanediol, such as the product marketed under the trademark Acnacidol® BG by Vincience; and

mixtures thereof.

Preferred anti-seborrhoeic active agents include:

benzoyl peroxide and vitamin B6 (or pyridoxine),

zinc salts such as zinc gluconate, zinc pyrrolidonecarboxylate (or zinc pidolate), zinc lactate, zinc aspartate, zinc carboxylate, zinc salicylate and zinc cysteate;

meadowsweet (Spiraea ulmaria) extracts, such as the product marketed under the trademark Sebonormine® by Silab;

extracts of the alga Laminaria saccharina, such as the product marketed under the trademark Phlorogine® by Biotechmarine;

mixtures of extracts of salad burnet root (Sanguisorba officinalis/Poterium officinale), of ginger rhizomes (Zingiber officinalis) and of cinnamon bark (Cinnamomum cassia), such as the product marketed under the trademark Sebustop® by Solabia;

clove extract, such as the product marketed under the trademark Clove extract powder by Maruzen;

lactic protein filtrates, such as the product marketed under the trademark Normaseb® by Sederma;

European meadowsweet (Spiraea ulmaria) extracts, such as the product marketed under the trademark Cytobiol® Ulmaire by Libiol;

sebacic acid, especially marketed in the form of a sodium polyacrylate gel under the trademark Sebosoft® by Sederma;

glycine grafted onto an undecylenic chain, such as the product marketed under the trademark Lipacide UG OR by SEPPIC;

tri(C₁₂-C₁₃)alkyl citrate marketed under the trademark Cosmacol® ECI by Sasol; tri(C₁₄-C₁₅)alkyl citrate marketed under the trademark Cosmacol® ECL by Sasol;

10-hydroxydecanoic acid, and especially mixtures of 10-hydroxydecanoic acid, of sebacic acid and of 1,10-decanediol, such as the product marketed under the trademark Acnacidol® BG by Vincience; and

mixtures thereof.

Preferentially, the anti-seborrhoeic active agent is selected from:

zinc salts such as zinc gluconate, zinc pyrrolidonecarboxylate (or zinc pidolate), zinc lactate, zinc aspartate, zinc carboxylate, zinc salicylate and zinc cysteate; and preferably zinc pyrrolidonecarboxylate (or zinc pidolate) or zinc salicylate;

clove extract, such as the product marketed under the trademark Clove extract powder by Maruzen;

glycine grafted onto an undecylenic chain, such as the product marketed under the trademark Lipacide UG OR by SEPPIC;

tri(C₁₂-C₁₃)alkyl citrate marketed under the trademark Cosmacol® ECI by Sasol; tri(C₁₄-C₁₅)alkyl citrate marketed under the trademark Cosmacol® ECL by Sasol;

and mixtures thereof.

The anti-seborrhoeic active agent is, for example, present in a content ranging from 0.1% to 10% by weight, preferably from 0.1% to 5% by weight and preferentially from 0.5% to 3% by weight relative to the total weight of the composition.

Astringents:

According to the invention, the term “astringents” means agents for combating the dilation of the sebaceous follicles.

As astringents that may be included in the compositions according to the invention, exemplary are extracts of mushroom pulp (Polyporus officinalis), for instance Laricyl LS8865® from Cognis, extracts of Terminalia catappa and Sambucus nigra, for instance Phytofirm LS9120® from Cognis, extracts of gall nut, for instance Tanlex VE® from Ichimaru Pharcos, aluminum hydroxychloride, centella extracts (e.g. Plantactiv centella from Cognis), dicetyl dimethylammonium chloride, for instance Varisoft 432 CG® from Degussa, common horsechestnut extracts, mallow extracts, witch-hazel extracts, sweet almond extracts, marsh mallow root extracts and linseed extracts, for instance Almondermin LS 3380® from Cognis, burdock extracts, nettle extracts, birch extracts, horsetail extracts, camomile extracts, for instance those marketed under the trademark Extrapone 9 Special® by Symrise, skullcap extracts, European meadowsweet extracts (for example Cytobiol Ulmaire from Libiol), a mixture of extracts of white ginger, of horsetail, of nettle, of rosemary and of yucca, for instance Herb extract B1348® from Bell Flavors & Fragrances, extracts of acacia, of elm, of white willow, of cinnamon, of birch and of meadowsweet, Panama sapogenins, zinc phenolsulfonate from Interchemical, extracts of gentian, of cucumber and of walnut, the mixture of extracts of Ratanhia, of grapefruit, of gumweed and of oak gall, for instance Epilami® from Alban Muller.

Preferred astringents according to the invention, include skullcap extracts, European meadowsweet extracts, meadowsweet extracts, gentian extracts and burdock extracts, and mixtures thereof.

Cicatrizing Agents:

Examples of cicatrizing agents include:

allantoin, urea, certain amino acids, for instance hydroxyproline, arginine, and serine, and also extracts of white lily (for instance Phytélène Lys 37EG 16295 from Indena), a yeast extract, for instance the cicatrizing agent LS LO/7225B from Laboratoires Serobiologiques), tamanu oil, extract of Saccharomyces cerevisiae, for instance Biodynes® TRF® from Arch Chemical, oat extracts, chitosan and derivatives, for instance chitosan glutamate, carrot extracts, artemia extract, for instance GP4G® from Vincience, sodium acexamate, lavandin extracts, propolis extracts, ximeninic acid and salts thereof, rose hip oil, marigold extracts, for instance Souci Ami® Liposolible from Alban Muller, horsetail extracts, lemon peel extracts, for instance Herbasol® citron from Cosmetochem, helichrysum extracts, common yarrow extracts and folic acid.

Preferred cicatrizing agents according to the invention include arginine, serine, folic acid, tamanu oil, sodium acexamate, horsetail extracts and helichrysum extracts, and mixtures thereof.

Anti-Inflammatory Agents:

As particular anti-inflammatory agents according to the invention, exemplary are cortisone, hydrocortisone, indomethacin, betamethasone, azelaic acid, acetaminophen, diclofenac, clobetasol propionate, folic acid; an extract of Eperua falcata bark, such as the product marketed by Cognis under the trademark Eperuline®; an extract of Paeonia suffruticosa root, such as the product marketed by Ichimaru Pharcos under the trademark Botanpi Liquid B®; and mixtures thereof.

Preferred anti-inflammatory agents are azelaic acid, folic acid, an extract of Eperua falcata bark, such as the product marketed by Cognis under the trademark Eperuline®; an extract of Paeonia suffruticosa root, such as the product marketed by Ichimaru Pharcos under the trademark Botanpi Liquid B®; and mixtures thereof.

Anti-Acne Agents:

In one advantageous embodiment of the invention, the composition may also comprise at least one anti-acne active.

The term “anti-acne active” especially means any active agent that has effects on the specific flora of greasy skin, for instance Propionibacterium acnes (P. acnes).

These effects may be bactericidal.

Antibacterial actives that are exemplary include:

actives and preservatives with antimicrobial activity mentioned in DE 103 24 567, which is incorporated into the present invention by reference,

asiatic acid,

the monoethanolamine salt of 1-hydroxy-4-methyl 6-trimethylpentyl-2-pyridone (INCI name: piroctone olamine), marketed especially under the trademark Octopirox® by Clariant;

citronellic acid, perillic acid (or 4-isopropenylcyclohex-1-enecarboxylic acid),

glyceryl 2-ethylhexyl ether (INCI name: ethylhexylglycerine), for example marketed under the trademark Sensiva SC 50® by Schulke & Mayr,

glyceryl caprylate/caprate, for example marketed under the trademark Capmul MCM® by Abitec;

sodium calcium phosphosilicate, especially marketed under the trademarks Bioactive Glasspowder® and Actysse Premier BG® by Schott Glass;

silver-based particles, for example those marketed under the trademark Metashine ME 2025 PS® by Nippon Sheet Glass;

hop cone extract (Humulus lupulus) obtained by supercritical CO₂ extraction, such as the product marketed under the trademark HOP CO2-TO Extract® by Flavex Naturextrakte,

St. John's Wort extract obtained by supercritical CO₂ extraction, such as the product marketed under the trademark St. John's Wort CO2-TO Extract® by Flavex Naturextrakte,

the mixture of extracts of roots of Scutellaria baicalensis, of Paeonia suffruticosa and Glycyrrhiza glabra, such as the product marketed under the trademark BMB—CF® by Naturogin,

argan tree extract, for instance Argapure LS9710® from Cognis;

bearberry leaf extracts, for instance the product marketed under the trademark Melfade-J by Pentapharm;

10-hydroxy-2-decanoic acid such as Acnacidol P® from Vincience, sodium ursolate, azelaic acid, diiodomethyl p-tolyl sulfone such as Amical Flowable® from Angus, malachite powder, zinc oxide such as Zincare® from Elementis GMBH, octadecenedioic acid such as Arlatone dioic DCA® from Uniqema; ellagic acid; 2,4,4′-trichloro-2′-hydroxydiphenyl ether (or triclosan), 1-(3′,4′-dichlorophenyl)-3-(4′-chlorophenyl)urea (or triclocarban), 3,4,4′-trichlorocarbanilide, 3′,4′,5′-trichlorosalicylanilide, phenoxyethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and salts thereof, benzoyl peroxide, 3-hydroxybenzoic acid, 4-hydroxybenzoic acid, phytic acid, N-acetyl-L-cysteine, lipoic acid, azelaic acid and salts thereof, arachidonic acid, resorcinol, 3,4,4′-trichlorocarbanalide, octoxyglycerine or octoglycerine, octanoylglycine such as Lipacid C8G® from SEPPIC, caprylyl glycol, 10-hydroxy-2-decanoic acid, dichlorophenylimidazoldioxolane and derivatives thereof described in WO 93/18743, iodopropynyl butylcarbamate, 3,7,11-trimethyldodeca-2,5,10-trienol or farnesol, phytosphingosines; quaternary ammonium salts, for instance cetyltrimethylammonium salts and cetylpyridinium salts, and

mixtures thereof.

Also exemplary are certain surfactants with an antimicrobial effect, for instance sodium cocoamphoacetate or disodium diacetate such as Miranol C2M Conc. NP, betaines, for instance the cocoyl betaine Genagen KB from Clariant, sodium lauryl ether sulfate, for instance Emal 270 D from Kao, decyl glucoside, for instance Plantacare 2000 UP, branched C₁₂₋₁₃ dialkyl malates, for instance Cosmacol EMI, propylene glycol monoesters, for instance propylene glycol monolaurate, monocaprylate or monocaprate, lauryldimethylamine betaine, for instance Empigen BB/LS, and also polyquaternary ammoniums such as Quaternium-24 or Bardac 2050 from Lonza and those described in FR 0 108 283, and mixtures thereof.

Preferred antimicrobial agents are octoglycerine or octoxyglycerine, and 10-hydroxy-2-decanoic acid, and mixtures thereof.

Other additional anti-acne actives may be added to the abovementioned anti-acne actives.

Especially exemplary are actives with bacterial anti-adhesion effects or agents that act on the biofilm of bacteria to prevent them from multiplying.

As agents for preventing and/or reducing the adhesion of microorganisms, especially exemplary are:

phytantriol and derivatives thereof as described in EP-1-529,523, plant oils such as wheatgerm oil, calendula oil, castor oil, olive oil, avocado oil, sweet almond oil, groundnut oil, jojoba oil, sesame seed oil, apricot kernel oil, sunflower oil and macadamia oil, described in EP-1-133,979, or certain surfactants such as disodium cocoamphodiacetate, oxyethylenated (7 EO) glyceryl cocoate, 18-hexadecenyl succinate, octoxyglyceryl palmitate, octoxyglyceryl behenate, dioctyl adipate, PPG-15 stearyl ether, and the branched C₁₂-C₁₃ dialkyl tartrates described in EP-1-129,694, and mixtures thereof.

In particular with regard to the propagation of P. acnes, or as active agents that act on the biofilm of bacteria to prevent them from proliferating, exemplary are pentylene glycol, Nylon-66 (polyamide 66 fibers), rice bran oil, polyvinyl alcohol such as Celvol 540 PV Alcohol® from Celanese Chemical, rapeseed oil such as Akorex L® from Karlshamns, and fructose derivatives, and mixtures thereof.

The anti-acne active may be present in a content ranging from 0.01% to 10% by weight and preferably from 0.05% to 5% by weight relative to the total weight of the composition.

As a function of the nature and/or solubility of the abovementioned active agents, one skilled in this art will know how to select the most suitable embodiment according to the invention.

As lipophilic active agents that may be used in the kit or at least one of the compositions of the invention, especially exemplary are D-α-tocopherol, DL-α-tocopherol, D-α-tocopheryl acetate, DL-α-tocopheryl acetate, ascorbyl palmitate, vitamin F glycerides, D vitamins, vitamin D2, vitamin D3, retinol, retinol esters, retinyl palmitate, retinyl propionate, carotenes including β-carotene, D-panthenol, farnesol, farnesyl acetate, salicylic acid and derivatives thereof, for instance 5-n-octanoylsalicylic acid, α-hydroxy acid alkyl esters such as citric acid, lactic acid, glycolic acid, asiatic acid, madecassic acid, asiaticoside, the total extract of Centella asiatica, β-glycyrrhetinic acid, α-bisabolol, ceramides, for instance 2-oleoylamino-1,3-octadecane, phytantriol, phospholipids of marine origin rich in polyunsaturated essential fatty acids, ethoxyquine, rosemary extract, balm extract, quercetin, extract of dried microalgae, essential oil of bergamot, octyl methoxycinnamate, butylmethoxydibenzoylmethane, octyl triazone, 3,5-di-tert-butyl-4-hydroxy-3-benzylidenecamphor, antibiotics, antifungal agents, anaesthetics, analgesics, antiseptics, antiviral agents, pesticides and herbicides, and mixtures thereof.

The cosmetic and/or dermatological active agents will be present in the kit or one of the compositions according to the invention in a content ranging from 0.001% to 20% relative to the total weight of the composition, preferably from 0.01% to 10%, even more preferentially from 0.5% to 5% to more preferably from 0.1% to 1% by weight relative to the total weight of the composition.

For peeling applications, the contents of cosmetic and/or dermatological active agents may range from 1% to 50% by weight relative to the total weight of the composition and preferably from 1% to 30% by weight relative to the total weight of the composition.

Peels are a well-known means for improving the appearance and/or texture of the skin and/or the scalp, especially for improving the radiance and homogeneity of the complexion and/or for reducing the visible and/or tactile irregularities of the skin, and in particular for improving the surface appearance of the skin, for attenuating actinic lentigo, acne or chicken pox marks, and also for preventing, attenuating or combating the signs of aging of the skin, and especially for smoothing out irregularities in the texture of the skin, such as wrinkles and fine lines.

They have the effect of removing a surface part of the skin to be treated (epidermis and possibly the upper layer of the dermis), via chemical methods.

Other Additional Ingredients:

To complement and/or optimize the effects imparted by the cosmetic and/or dermatological actives mentioned above on the keratin materials, it may be advantageous to incorporate into the compositions of the invention other additional ingredients.

In particular, these additional ingredients may impart an immediate visual effect that will be taken up by the biological effect of the actives mentioned above. They may also, via a mechanical action (e.g.: abrasive fillers), amplify the effect of the biological actives mentioned above.

Thus, the compositions according to the invention may further comprise at least one agent selected from matting agents, soft-focus effect fillers, fluorescers, agents for promoting the naturally pinkish coloration of the skin, abrasive fillers or exfoliants, and mixtures thereof.

Matting Agents:

The term “matting agent” means agents intended to make the skin visibly more matt and less shiny.

The matting effect of the agent and/or composition containing it may especially be evaluated using a gonioreflectometer, by measuring the ratio R from the specular reflection and the scattered reflection. A value of R of less than or equal to 2 generally reflects a matting effect.

The matting agent may especially be selected from a rice starch or a corn starch, kaolinite, talc, a pumpkin seed extract, cellulose microbeads, plant fibers, synthetic fibers, in particular polyamide fibers, expanded acrylic copolymer microspheres, polyamide powders, silica powders, polytetrafluoroethylene powders, silicone resin powders, acrylic polymer powders, wax powders, polyethylene powders, powders of elastomeric crosslinked organopolysiloxane coated with silicone resin, talc/titanium dioxide/alumina/silica composite powders, amorphous mixed silicate powders, silicate particles and especially mixed silicate particles, and mixtures thereof.

Examples of matting agents that are especially representative include:

rice or corn starch, in particular an aluminum starch octenyl succinate marketed under the trademark Dry Flo® by National Starch;

kaolinite;

silicas;

talc;

a pumpkin seed extract as marketed under the trademark Curbilene® by Indena;

cellulose microbeads as described in EP-1-562,562;

fibers, such as silk fiber, cotton fiber, wool fiber, flax fiber, cellulose fiber extracted especially from wood, from vegetables or from algae, polyamide fiber (Nylon®), modified cellulose fiber, poly-p-phenyleneterephthamide fiber, acrylic fiber, polyolefin fiber, glass fiber, silica fiber, aramid fiber, carbon fiber, Teflon® fiber, insoluble collagen fiber, polyester fiber, polyvinyl chloride or polyvinylidene chloride fiber, polyvinyl alcohol fiber, polyacrylonitrile fiber, chitosan fiber, polyurethane fiber, polyethylene phthalate fiber, fibers formed from a mixture of polymers, resorbable synthetic fibers, and mixtures thereof described in EP-1-151,742;

expanded acrylic copolymer microspheres such as those marketed by EXPANCEL under the trademark Expancel 551®;

fillers with an optical effect as described in FR 2 869 796, in particular:

polyamide powders (Nylon®), for instance Nylon 12 particles of the Orgasol type from Arkema, with a mean size of 10 microns and a refractive index of 1.54,

silica powders, for instance Silica beads SB150 from Miyoshi with a mean size of 5 microns and a refractive index of 1.45,

polytetrafluoroethylene powders, for instance PTFE Ceridust 9205F from Clariant, with a mean size of 8 microns and a refractive index of 1.36,

silicone resin powders, for instance the silicone resin Tospearl 145A from GE Silicone with a mean size of 4.5 microns and a refractive index of 1.41,

acrylic copolymer powders, especially of polymethyl(meth)acrylate, for instance the PMMA particles Jurymer MBI from Nihon Junyoki, with a mean size of 8 microns and a refractive index of 1.49, or the Micropearl M100® and F 80 ED® particles by Matsumoto Yushi-Seiyaku,

wax powders, for instance the paraffin wax particles Microease 114S from Micropowders, with a mean size of 7 microns and a refractive index of 1.54,

polyethylene powders, especially comprising at least one ethylene/acrylic acid copolymer, and in particular consisting of ethylene/acrylic acid copolymers, for instance the particles Flobeads EA 209 from Sumitomo (with a mean size of 10 microns and a refractive index of 1.48),

elastomeric crosslinked organopolysiloxane powders coated with silicone resin, especially with silsesquioxane resin, as described, for example, in U.S. Pat. No. 5,538,793. Such elastomeric powders are marketed under the trademarks KSP-100, KSP-101, KSP-102, KSP-103, KSP-104 and KSP-105 by Shin-Etsu, and

talc/titanium dioxide/alumina/silica composite powders such as those marketed under the trademark Coverleaf® AR-80 by Catalyst & Chemicals,

mixtures thereof,

compounds that absorb and/or adsorb sebum as described in FR 2 869 796. Mention may be made especially of:

silica powders, for instance the porous silica microspheres marketed under the trademark Silica Beads SB-700 marketed by Miyoshi, the products Sunsphere® H51, Sunsphere® H33 and Sunsphere® H53 marketed by Asahi Glass; the polydimethylsiloxane-coated amorphous silica microspheres marketed under the trademark SA Sunsphere® H-33 and SA Sunsphere® H-53 marketed by Asahi Glass;

amorphous mixed silicate powders, especially of aluminum and magnesium, for instance the product marketed under the trademark Neusilin UFL2 by Sumitomo;

polyamide (Nylon®) powders, for instance Orgasol® 4000 marketed by Arkema, and

acrylic polymer powders, especially of polymethyl methacrylate, for instance Covabead® LH85 marketed by Wacker; of polymethyl methacrylate/ethylene glycol dimethacrylate, for instance Dow Corning 5640 Microsponge® Skin Oil Adsorber marketed by Dow Corning, or Ganzpearl® GMP-0820 marketed by Ganz Chemical; of polyallyl methacrylate/ethylene glycol dimethacrylate, for instance Poly-Pore® L200 or Poly-Pore® E200 marketed by Amcol; of ethylene glycol dimethacrylate/lauryl methacrylate copolymer, for instance Polytrap® 6603 marketed by Dow Corning;

silicate particles, such as alumina silicate;

mixed silicate particles, such as:

magnesium aluminum silicate particles, such as saponite or hydrated magnesium aluminum silicate with a sodium sulfate marketed under the trademark Sumecton® by Kunimine;

the magnesium silicate, hydroxyethylcellulose, black cumin oil, marrow oil and phospholipids complex or Matipure® from Lucas Meyer, and

mixtures thereof.

Preferred matting agents according to the invention include a pumpkin seed extract, a rice or corn starch, kaolinite, silicas, talc, polyamide powders, polyethylene powders, acrylic copolymer powders, expanded acrylic copolymer microspheres, silicone resin microbeads and mixed silicate particles, and mixtures thereof.

Fillers with a Soft-Focus Effect:

These fillers may be any material capable of modifying and hiding wrinkles by virtue of their intrinsic physical properties. These fillers may especially modify wrinkles via a tensioning effect, a covering effect or a soft-focus effect.

Examples of such fillers include the following compounds:

porous silica microparticles, for instance Silica Beads® SB150 and SB700 from Miyoshi with a mean size of 5 μm; the series-H Sunspheres® from Asahi Glass, for instance Sunspheres H33, H51 with respective sizes of 3.5 and 5 μm;

hollow hemispherical silicone resin particles such as NLK 500®, NLK 506® and NLK 510® from Takemoto Oil and Fat, especially described in EP-A-1-579,849;

silicone resin powders, for instance the silicone resin Tospearl® 145A from GE Silicone, with a mean size of 4.5 μm;

acrylic copolymer powders, especially of polymethyl (meth)acrylate, for instance the PMMA particles Jurimer MBI® from Nihon Junyoki, with a mean size of 8 μm, the hollow PMMA spheres marketed under the trademark Covabead® LH85 by Wackherr, and vinylidene/acrylonitrile/methylene methacrylate expanded microspheres marketed under the trademark Expancel®;

wax powders, for instance the paraffin wax particles MicroEase® 114S from MicroPowders, with a mean size of 7 μm;

polyethylene powders, especially comprising at least one ethylene/acrylic acid copolymer, for instance the Flobeads® EA 209 from Sumitomo, with a mean size of 10 μm;

crosslinked elastomeric organopolysiloxane powders coated with silicone resin and especially with silsesquioxane resin, marketed under the trademarks KSP-100®, KSP-101®, KSP-102®, KSP-103®, KSP-104® and KSP-105® by Shin-Etsu;

talc/titanium dioxide/alumina/silica composite powders, for instance Coverleaf AR 80® by Catalyst & Chemical;

talc, mica, kaolin, lauryl glycine, starch powders crosslinked with octenyl succinate anhydride, boron nitride, polytetrafluoroethylene powders, precipitated calcium carbonate, magnesium carbonate, magnesium hydrogen carbonate, barium sulfate, hydroxyapatite, calcium silicate, cerium dioxide and glass or ceramic microcapsules;

hydrophilic or hydrophobic, synthetic or natural, mineral or organic fibers such as silk fibers, cotton fibers, wool fibers, flax fibers, cellulose fibers extracted especially from wood, vegetables or algae, polyamide (Nylon®) fibers, modified cellulose fibers, poly-p-terephthamide fibers, acrylic fibers, polyolefin fibers, glass fibers, silica fibers, aramid fibers, carbon fibers, polytetrafluoroethylene (Teflon®) fibers, insoluble collagen fibers, polyester fibers, polyvinyl chloride fibers, polyvinylidene chloride fibers, polyvinyl alcohol fibers, polyacrylonitrile fibers, chitosan fibers, polyurethane fibers, polyethylene phthalate fibers, fibers formed from a mixture of polymers, resorbable synthetic fibers, and mixtures thereof described in EP-1-151,742;

spherical elastomeric crosslinked silicones, for instance Trefil E-505C® or E-506C® from Dow Corning;

abrasive fillers, which, via a mechanical effect, smooth out the skin microrelief, such as abrasive silica, for instance Abrasif SP® from Semanez or nutshell powders (for example of apricot or walnut, from Cosmetochem).

The fillers with an effect on the signs of aging are especially selected from porous silica microparticles, hollow hemispherical silicone particles, silicone resin powders, acrylic copolymer powders, polyethylene powders, crosslinked elastomeric organopolysiloxane powders coated with silicone resin, talc/titanium dioxide/alumina/silica composite powders, precipitated calcium carbonate, magnesium carbonate, magnesium hydrogen carbonate, barium sulfate, hydroxyapatite, calcium silicate, cerium dioxide, glass or ceramic microcapsules, and silk fibers or cotton fibers, and mixtures thereof.

The filler may be a soft-focus filler.

The term “soft-focus” filler means a filler which in addition gives the complexion transparency and a hazy effect. Preferably, the soft-focus fillers have a mean particle size of less than or equal to 15 microns. These particles may be in any form and in particular may be spherical or non-spherical. These fillers are more preferably non-spherical.

The soft-focus fillers may be selected from silica and silicate powders, especially alumina powder, powders of polymethyl methacrylate (PMMA) type, talc, silica/TiO₂ or silica/zinc oxide composites, polyethylene powders, starch powders, polyamide powders, styrene/acrylic copolymer powders and silicone elastomers, and mixtures thereof.

Particularly exemplary is a talc with a number-average size of less than or equal to 3 microns, for example talc with a number-average size of 1.8 microns and especially the product marketed under the trademark Talc P3® by Nippon Talc, Nylon® 12 powder, especially the product marketed under the trademark Orgasol 2002 Extra D Nat Cos® by Atochem, silica particles 1% to 2% surface-treated with a mineral wax (INCI name: hydrated silica (and) paraffin) such as the products marketed by Degussa, amorphous silica microspheres, such as the products marketed under the trademark Sunsphere, for example of reference H-530 by Asahi Glass, and silica microbeads such as those marketed under the trademark SB-700® or SB-150® by Miyoshi, this list not being limiting.

The concentration of these fillers with an effect on the signs of aging in the compositions according to the invention may be from 0.1% to 40%, or even from 0.1% to 20% by weight, relative to the total weight of the composition.

Fluorescers:

The term “fluorescer” means a substance which, under the effect of ultraviolet rays and/or visible light, re-emits in the visible region the portion of light that it has absorbed under the same color as that which it naturally reflects. The naturally reflected color is thus reinforced by the re-emitted color and appears extremely bright.

Examples thereof include colored polyamide and/or formaldehyde/benzoguanamine and/or melamine/formaldehyde/sulfonamide resins, from colored aminotriazine/formaldehyde/sulfonamide co-condensates and/or from metallized polyester flakes and/or mixtures thereof. These fluorescent pigments may also be present in the form of aqueous dispersions of fluorescent pigments.

Also exemplary are pink-colored fluorescent aminotriazine/formaldehyde/sulfonamide co-condensate with a mean particle size of 3-4 microns marketed under the trademark “Fiesta Astral Pink FEX-1” and the blue-colored fluorescent aminotriazine/formaldehyde/sulfonamide co-condensate with a mean particle size of 3-4.5 microns marketed under the trademark “Fiesta Comet Blue FTX-60” by Swada, or, alternatively, the yellow-colored benzoguanamine/formaldehyde resin covered with formaldehyde/urea resin marketed under the trademark “FB-205 Yellow” and the red-colored benzoguanamine/formaldehyde resin covered with formaldehyde/urea resin marketed under the trademark “FB-400 Orange Red” by UK Seung Chemical, and the orange-colored polyamide resin marketed under the trademark “Flare 911 Orange 4” by Sterling Industrial Colors.

The fluorescent substances are preferably present in the composition in a content ranging from 0.1% to 20%, preferably from 0.1% to 15% to more preferably from 0.5% to 3% by weight relative to the total weight of the composition.

When the organic fluorescent substances are white, they are also known as optical brighteners.

The optical brightener has the effect of intensifying the radiance and reviving the shades of cosmetic compositions comprising them on application to the skin.

Among the optical brighteners that are more particularly exemplary are stilbene derivatives, in particular polystyrylstilbenes and triazinestilbenes, coumarin derivatives, in particular hydroxycoumarins and aminocoumarins, oxazole, benzoxazole, imidazole, triazole and pyrazoline derivatives, pyrene derivatives and porphyrin derivatives, and/or mixtures thereof.

Such compounds are available, for example, under the trademarks Tinopal SOP® and Uvitex OB® by Ciba Geigy.

The optical brighteners preferentially used are sodium 4,4′-bis[(4,6-dianilino-1,3,5-triazin-2-yl)amino]stilbene-2,2′-disulfonate, 2,5-thiophenediylbis(5-tert-butyl-1,3-benzoxazole) and disodium 4,4′-distyrylbiphenylsulfonate, and/or mixtures thereof.

Agents for Promoting the Naturally Pinkish Coloration of the Skin:

Especially exemplary are:

a self-tanning agent, i.e., an agent which, when applied to the skin, especially to the face, can produce a tan effect that is more or less similar in appearance to that which may result from prolonged exposure to the sun (natural tan) or under a UV lamp;

an additional coloring agent, i.e., any compound that has a particular affinity for the skin, which allows it to give the skin a lasting, non-covering coloration (i.e., that does not have a tendency to opacify the skin) and that is not removed either with water or using a solvent, and that withstands both rubbing and washing with a solution containing surfactants. Such a lasting coloration is thus distinguished from the superficial and transient coloration provided, for example, by a makeup pigment; and mixtures thereof.

Examples of self-tanning agents include:

dihydroxyacetone (DHA),

erythrulose, and

the combination of a catalytic system formed from:

manganese and/or zinc oxide salts, and

alkali metal and/or alkaline-earth metal hydrogen carbonates.

The self-tanning agents are generally selected from monocarbonyl or polycarbonyl compounds, for instance isatin, alloxan, ninhydrin, glyceraldehyde, mesotartaric aldehyde, glutaraldehyde, erythrulose, pyrazoline-4,5-dione derivatives as described in FR 2,466,492 and WO 97/35842, dihydroxyacetone (DHA) and 4,4-dihydroxypyrazolin-5-one derivatives as described in EP-903,342. DHA will preferably be used.

The DHA may be used in free and/or encapsulated form, for example in lipid vesicles such as liposomes, especially described in WO 97/25970.

In general, the self-tanning agent is present in an amount ranging from 0.01% to 20% by weight and preferably in an amount of from 0.1% to 10% of the total weight of the composition.

Other dyes that allow modification of the color produced by the self-tanning agent may also be used.

These dyes may be selected from synthetic or natural direct dyes.

These dyes may be selected, for example, from red or orange dyes of the fluoran type such as those described in FR 2,840,806.

Exemplary are the following dyes:

tetrabromofluoresceine or eosin known under the CTFA name: CI 45380 or Red 21;

phloxin B known under the CTFA name: CI 45410 or Red 27;

diiodofluoresceine known under the CTFA name: CI 45425 or Orange 10;

dibromofluoresceine known under the CTFA name: CI 45370 or Orange 5;

the sodium salt of tetrabromofluoresceine known under the CTFA name: CI 45380 (Na salt) or Red 22;

the sodium salt of phloxin B known under the CTFA name: CI 45410 (Na salt) or Red 28;

the sodium salt of diiodofluoresceine known under the CTFA name: CI 45425 (Na salt) or Orange 11;

erythrosine known under the CTFA name: CI 45430 or Acid Red 51;

phloxin known under the CTFA name: CI 45405 or Acid Red 98.

These dyes may also be selected from anthraquinones, caramel, carmine, carbon black, azulene blues, methoxalene, trioxalene, guajazulene, chamuzulene, Bengal rose, cosin 10B, cyanosin and daphinin.

These dyes may also be selected from indole derivatives, for instance the monohydroxyindoles as described in FR 2 651 126 (i.e., 4-, 5-, 6- or 7-hydroxyindole) or the dihydroxyindoles as described in EP-B-0,425,324 (i.e., 5,6-dihydroxyindole, 2-methyl-5,6-dihydroxyindole, 3-methyl-5,6-dihydroxyindole or 2,3-dimethyl-5,6-dihydroxyindole).

Abrasive Fillers or Exfoliants:

As exfoliants that may be included in rinse-out compositions according to the invention, examples thereof include exfoliant or scrubbing particles of mineral, plant or organic origin. Thus, polyethylene beads or powder, Nylon powder, polyvinyl chloride powder, pumice powder, ground apricot kernel or walnut shell, sawdust, glass beads and alumina, and mixtures thereof, may be used, for example.

Also exemplary are Exfogreen® from Solabia (bamboo extract), extracts of strawberry akenes (Strawberry Akenes from Greentech), peach kernel powder, apricot kernel powder, and finally, in the field of plant powders with an abrasive effect, mention may be made of cranberry kernel powder.

As abrasive fillers or exfoliants that are preferred according to the invention, exemplary are peach kernel powder, apricot kernel powder, cranberry kernel powder, strawberry akene extracts and bamboo extracts.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES 1 AND 2

Vaporizable fluid anti-sun/sunscreen formulations containing the following ingredients were prepared.

| Ingredients | Ex. 1 | Ex. 2 |
|---|---|---|
| Ethylhexyl salicylate (Neo Heliopan OS) | 5.0 | 5.0 |
| Butylmethoxydibenzoylmethane (Parsol 1789 - DSM) | 4.0 | 4.0 |
| Octocrylene (Uvinul N539 - BASF) | 3.5 | 3.5 |
| bis-Ethylhexyloxyphenol methoxyphenyl triazine | 3.0 | 3.0 |
| (Tinosorb S - Ciba) | | |
| Ethylhexyl triazone (Uvinul T150 - BASF) | 2.5 | 2.5 |
| Terephthalylidenedicamphorsulfonic acid | 1.0 | 3.0 |
| (Mexoryl SX - Chimex) | | |
| Drometrizole trisiloxane (Mexoryl X - Chimex) | 1.5 | 1.5 |
| Titanium dioxide (Microtitanium Dioxide | 3.5 | 3.5 |
| MT 100 AQ - Tayca) | | |
| Denatured ethyl alcohol | 4.3 | 4.3 |
| C₁₂-C₁₅ alkyl benzoate | 12.5 | 12.5 |
| Glycerol | 6 | 6 |
| Tocopherol (and) soya glycine | 0.2 | 0.2 |
| Pentasodium salt of ethylenediaminetetra- | 0.3 | 0.3 |
| methylenephosphonic acid at 33% in water | | |
| (Dequest 2046 - Solutia) | | |
| Propylene glycol | 6.0 | 6.0 |
| Ethylhexyl glycerin (Sensitiva SC 50 - Schulke & | 0.5 | 0.5 |
| Mayr) | | |
| Polyester-5 (Eastman AQ 38S - Eastman Chemical) | 2.0 | 2.0 |
| Ethylenediamide/hydrogenated dimer dilinoleate | 1.5 | — |
| copolymer bis-di-C₁₄₋₁₈ alkyl amide (Sylvaclear | | |
| A200V - Arizona Chemical) | | |
| Polyacrylate-3 as a 25% emulsion in water | 0.7 | 0.7 |
| (Viscophobe DB 1000 - Amerchol) | | |
| Triethanolamine | 0.7 | 0.66 |
| Demineralized water | qs 100 | qs 100 |

The composition does not fluff when applied to the skin.

A comparative anti-sun formulation B of the same composition as formulation A, but not containing any ATPA polymer, was then prepared.

The viscosities of compositions 1 and 2, measured using a Rheomat 180 viscometer at 25° C. at a rotation speed of 200 rpm after 10 minutes, are, respectively, 0.35 Pa·s and 0.31 Pa·s.

For each of the compositions 1 and 2, the sun protection factor (SPF) associated therewith was then determined. This was determined by using the in vitro method described by V. Wandel et al. in SÖFW Journal 127 (2001); this method consists in determining the monochromatic protection factors over a wavelength range from 290 to 400 nm and in calculating therefrom the sun protection factor according to a given mathematical equation. The measurement was performed with a 1 nm interval on a UV-1000S machine by Labsphere, 0.6 mg/cm² of product being spread onto a frosted PMMA plate. The results (mean value corresponding to 5 plates per product, 10 points per plate) are collated in Table (I) below:

TABLE (I)

| | 1 (invention) with | 2 (outside the invention) without |
|---|---|---|
| Composition | ATPA polymer | ATPA polymer |
| Mean SPF | 78.9 (1.6) | 60.4 (11.8) |
| (standard | | |
| deviation) | | |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable, fluid anti-sun/sunscreen composition useful for photoprotecting the skin and/or the hair against the damaging effects of ultraviolet radiation, comprising:
(a) at least one photoprotective system for screening out UV radiation; and
(b) at least one tertiary-amide-terminated polyamide (ATPA) polymer, formulated into a topically applicable, cosmetically acceptable aqueous support therefor.

2. The anti-sun/sunscreen composition as defined by claim 1, said at least one tertiary-amide-terminated polyamide polymer having the formula (1) below: 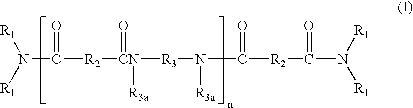
in which n is an integer greater than 0 which is a number of units such that the terminal amide groups containing R₁ constitute from 10% to 50% of the total number of amide groups in the formula (1);
the radicals R₁, which may be identical or different, are each a C₁-C₂₂ hydrocarbon group;
the radicals R₂, which may be identical or different, are each a C₂-C₄₂ hydrocarbon group, with the proviso that at least 50% of the radicals R₂ have 30 to 42 carbon atoms;
the radicals R₃, which may be identical or different, are each an organic group which has at least two carbon atoms in combination with hydrogen atoms and can additionally contain one or more oxygen or nitrogen atoms;
the radicals R₃ₐ, which may be identical or different, are each hydrogen, a C₁-C₁₀ alkyl radical, a direct bond with the radical R₃ or with another radical R₃ₐ to form, with the nitrogen atom bonded both to the radical R₃ and to the radical R₃ₐ, a heterocycle defined in part by R₃ₐ—N—R₃.

3. The anti-sun/sunscreen composition as defined by claim 2, wherein formula (I) n ranges from 1 to 10.

4. The anti-sun/sunscreen composition as defined by claim 1, wherein the terminal tertiary amide groups of said at least one polyamide polymer constitute from 10% to 50%, of the total number of amide functional groups.

5. The anti-sun/sunscreen composition as defined by claim 2, wherein said at least one tertiary-amide-terminated polyamide polymer comprises a mixture of polymers of formula (I) having different values of n.

6. The anti-sun/sunscreen composition as defined by claim 1, wherein said at least one tertiary-amide-terminated polyamide polymer has a weight-average molecular weight ranging from 500 to 10,000.

7. The anti-sun/sunscreen composition as defined by claim 2, wherein said at least one tertiary-amide-terminated polyamide polymer is prepared by reacting x equivalents of a dimer acid reactant of formula HOOC—R₂—COOH, y equivalents of a diamine reactant represented by the formula HN(R₃ₐ)—R₃—N(R₃ₐ)—H and z equivalents of a secondary monoamine reactant of formula R₁—NH—R₁, the equivalents x, y and z being other than 0.

8. The anti-sun/sunscreen composition as defined by claim 7, in which the dimer acid reactant comprises a C₃₋₆ hydrogenated linoleic acid.

9. The anti-sun/sunscreen composition as defined by claim 7 or 8, in which the diamine reactant comprises ethylenediamine.

10. The anti-sun/sunscreen composition as defined by claim 7, in which said secondary amine reactant comprises a (C₁₄-C₁₈)alkylamine or a (C₁₄-C₁₈)alkylamine mixture.

11. The anti-sun/sunscreen composition as defined by claim 1, wherein said at least one tertiary-amide-terminated polyamide polymer constitutes the polyamide ethylenediamide/hydrogenated dimer dilinoleate copolymer bis-di-C₁₄-C₁₈ alkyl amide.

12. The anti-sun/sunscreen composition as defined by claim 1, wherein said at least one tertiary-amide-terminated polyamide polymer is present therein in a maximum amount of 10% by weight relative to the total weight of the composition.

13. The anti-sun/sunscreen composition as defined by claim 12, wherein said at least one tertiary-amide-terminated polyamide polymer is present therein in amounts ranging from 0.5% to 5% by weight relative to the total weight of the composition.

14. The anti-sun/sunscreen composition as defined by claim 1, wherein said at least one photoprotective system comprises one or more hydrophilic, lipophilic or insoluble organic screening agents and/or one or more mineral pigments.

15. The anti-sun/sunscreen composition as defined by claim 14, wherein said at least one photoprotective system comprises at least one hydrophilic, lipophilic or insoluble organic screening agent.

16. The anti-sun/sunscreen composition as defined by claim 14, comprising at least one organic screening agent selected from the group consisting of cinnamic derivatives; anthranilates; salicylic derivatives; dibenzoylmethane derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzoazolyl derivatives; p-aminobenzoic acid (PABA) derivatives; methylenebis(hydroxyphenylbenzotriazole) derivatives; benzoxazole derivatives; screening polymers and screening silicones; α-alkylstyrene-derived dimers; 4,4-diarylbutadienes; merocyanin derivatives; and mixtures thereof.

17. The anti-sun/sunscreen composition as defined by claim 16, comprising at least one organic screening agent selected from the group consisting of:
Ethylhexyl methoxycinnamate,
Homosalate,
Ethylhexyl salicylate,
Butylmethoxydibenzoylmethane,
Octocrylene,
Phenylbenzimidazolesulfonic acid,
Benzophenone-3,
Benzophenone-4,
Benzophenone-5,
n-Hexyl 2-(4-diethylamino-2-hydroxybenzoyl)benzoate,
4-Methylbenzylidenecamphor,
Terephthalylidenedicamphorsulfonic acid,
Disodium phenyldibenzimidazoletetrasulfonate,
Ethylhexyl triazone,
Bis-ethylhexyloxyphenol methoxyphenyl triazine,
Diethylhexyl butamido triazone,
2,4,6-Tris(dineopentyl 4′-aminobenzalmalonate)-s-triazine,
2,4,6-Tris(diisobutyl 4′-aminobenzalmalonate)-s-triazine,
Methylenebis(benzotriazolyl)tetramethylbutylphenol,
Drometrizole trisiloxane,
Polysilicone-15,
Dineopentyl 4′-methoxybenzalmalonate,
1,1-Dicarboxy(2,2′-dimethylpropyl)-4,4-diphenylbutadiene,
2,4-Bis[5-(1-dimethylpropyl)benzoxazol-2-yl(4-phenyl)imino]-6-(2-ethylhexyl)imino-1,3,5-triazine,
and mixtures thereof.

18. The anti-sun/sunscreen composition as defined by claim 14, comprising pigments or nanopigments selected from among coated or uncoated metal oxides.

19. The anti-sun/sunscreen composition as defined by claim 18, in which the mineral agent(s) for screening out UV radiation is (are) selected from among coated or uncoated titanium oxide, iron oxide, zinc oxide, zirconium oxide or cerium oxide nanopigments.

20. The anti-sun/sunscreen composition as defined by claim 1, wherein said at least one photoprotective system is present therein in a content ranging from 0.1% to 40% by weight relative to the total weight thereof.

21. The anti-sun/sunscreen composition as defined by claim 1, further comprising at least one cosmetic adjuvant selected from the group consisting of fatty substances, organic solvents, ionic or nonionic, hydrophilic or lipophilic thickeners, softeners, humectants, opacifiers, stabilizers, emollients, silicones, antifoams, fragrances, preservatives, anionic, cationic, nonionic, zwitterionic or amphoteric surfactants, fillers, polymers, propellants, acidifying or basifying agents and any other ingredient usually employed in cosmetics and/or dermatology.

22. The anti-sun/sunscreen composition as defined by claim 1, formulated as a simple or complex emulsion; a cream gel; an aqueous gel; a lotion, or is packaged in an aerosol in the form of a mousse or spray, or is packaged in an atomizer.

23. The anti-sun/sunscreen composition as defined by claim 1, formulated as an oil-in-water or water-in-oil emulsion.

24. The anti-sun/sunscreen composition as defined by claim 23, formulated as an emulsion containing at least one isophthalic acid or sulfoisophthalic acid polymer.

25. The anti-sun/sunscreen composition as defined by claim 24, in which said at least isophthalic acid or sulfoisophthalic acid polymer comprises Polyester-5.

26. The anti-sun/sunscreen composition as defined by claim 1, having a viscosity measured using a Rheomat 180 viscometer at 25° C. at a rotation speed of 200 rpm after 10 minutes of rotation of from 0.01 to 2 Pa·s.

27. The anti-sun/sunscreen composition as defined by claim 1, further comprises at least one cosmetic or dermatological active agent selected from the group consisting of moisturizers, desquamating agents, agents for improving the barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, tensioning agents, lipid restructuring agents, slimming agents, agents for promoting the cutaneous microcirculation, calmatives and/or anti-irritants, sebo-regulating or anti-seborrhoeic agents, astringents, cicatrizing agents, anti-inflammatory agents, anti-acne agents, and mixtures thereof.

28. The anti-sun/sunscreen composition as defined by claim 1, further comprises at least one immediate visual effect active agent selected from among matting agents, fillers with a soft-focus effect, fluorescers, agents for promoting the naturally pinkish coloration of the skin, abrasive fillers or exfoliants, and mixtures thereof.

29. The anti-sun/sunscreen composition as defined by claim 1, formulated in vaporizable form.

30. A pressurization device comprising:
(A) at least one reservoir containing at least one vaporizable fluid composition as defined in claim 29, and
(B) means for placing said composition under pressure.

31. The device as defined by claim 30, comprising a non-aerosol pump.

32. The device as defined by claim 30, comprising an aerosol container or an aerosol pump.

33. The device as defined by claim 32, comprising a two-compartment aerosol container or aerosol pump.

34. A regime or regimen for UV photoprotecting the skin and/or the hair of an individual in need of such treatment, comprising topically applying thereon a thus effective amount of the anti-sun/sunscreen composition as defined by claim 1.

35. A process for enhancing the sun protection factor (SPF) of a UV photoprotective anti-sun/sunscreen composition, comprising formulating therewith a thus effective amount of at least one tertiary-amide-terminated polyamide (ATPA) polymer.

36. A process for reducing or eliminating the fluffing effect of a UV photoprotective anti-sun/screen composition when topically applied onto the skin and/or hair of an individual, comprising first formulating therewith a thus effective amount of at least tertiary-amide-terminated polyamide (ATPA) polymer.
